(19) United States
(12) Patent Application Publication
(10) Pub. No.: US 2008/0194462 A1
(43) Pub. Date: Aug. 14, 2008

(54) Methods and Compositions for Treatment of Autoimmune Diseases

(75) Inventors: Kai Wucherpfennig, Brookline, MA (US); James Rasmussen, Cambridge, MA (US); Bei Yu, West Roxbury, MA (US); Eric Zanelli, Sudbury, MA (US); Jack L. Strominger, Cambridge, MA (US)

Correspondence Address: ROPES & GRAY LLP, PATENT DOCKETING 39/41, ONE INTERNATIONAL PLACE, BOSTON, MA, 02110-2624, US

(73) Assignee: Peptimmune, Inc., Cambridge, MA (US)

(21) Appl. No.: 10/591,315

(22) PCT Filed: Sep. 15, 2005
(86) PCT No.: PCT/US2005/006822

§ 371 Date: Oct. 19, 2007

Related U.S. Application Data
(60) Provisional application No. 60/549,409, filed on Mar. 1, 2004.
(60) Provisional application No. 60/551,396, filed on Mar. 9, 2004.

(51) Int. Cl. A61K 38/28; A61K 38/16; G01N 33/566; A61P 3/10; A61P 37/00

(52) U.S. Cl. 514/4; 514/12; 435/7.21

(57) ABSTRACT

The present invention provides methods and compositions for treating autoimmune diseases and other unwanted immune reactions comprising administering a copolymer that binds to one or more HLA-DQ molecules and modulates DQ-restricted T cell responses. The copolymers are random copolymers of amino acids and copolymers comprising anchor residues to facilitate binding to the DQ binding pockets.

### BACKGROUND OF THE INVENTION

An autoimmune disease results when a host's immune response fails to distinguish foreign antigens from self molecules (autoantigens) thereby eliciting an aberrant immune response. The immune response towards self molecules in an autoimmune disease results in a deviation from the normal state of self-tolerance, which involves the destruction of T cells and B cells capable of reacting against autoantigens, which has been prevented by events that occur in the development of the immune system early in life. The cell surface proteins that play a central role in regulation of immune responses through their ability to bind and present processed peptides to T cells are the major histocompatibility complex (MHC) molecules (Rothbard et al. (1991) Annu. Rev. Immunol. 9:527).

Certain human leukocyte antigen (HLA) alleles occur in a higher frequency in individuals with particular diseases than in the general population. HLA locus encodes major histocompatibility complex (MHC) genes of human. MHC molecules exist in two forms, class I and class II, both encoded within a single gene complex. MHC genes are highly polymorphic: some loci have up to several hundred alleles in the human population (Hansen et al. 1993 In “Fundamental Immunology” Ed. Paul, W. E., Raven Press, New York, N.Y., p. 577).

Class I MHC molecules are 45 kDa transmembrane glycoproteins, noncovalently associated with another glycoprotein, the 12 kDa β-2 microglobulin. The latter is not inserted into the cell membrane, and is encoded outside the MHC region of the genome. Human class I molecules are of three different isotypes, termed HLA-A, -B, and -C, encoded in separate loci. The tissue expression of class I molecules is ubiquitous and codominant. The three-dimensional structure of several human and murine class I molecules have been resolved (Bjorkman et al. (1987) Nature 329: 506; Garrett et al. (1989) Nature 342: 692; Madden et al. (1991) Nature 353: 321; Fremont et al. (1992) Science 257: 919). The three class I isotypes, as well as their allelic forms, have different peptide binding specificities, depending on polymorphic residues within the binding site (Falk et al. (1991) Nature 351: 290; Falk et al. (1992) Eur. J. Immunol., 22: 277).

Class II MHC molecules are noncovalently associated heterodimers of two transmembrane glycoproteins, the 35 kDa α chain and the 28 kDa β chain. In humans, class II molecules occur as three different isotypes, termed HLA-DP, -DQ, and -DR. There are a minimum of six α and eight β genes, which are arranged in distinct clusters. Polymorphism in DR is restricted to the β chain, whereas both chains are polymorphic in the DP and DQ isotypes. Structural variation in class II gene products is linked to functional features of immune recognition, leading to individual variations in histocompatibility, immune recognition, and susceptibility to disease. Two types of dimers on the cell-surface are made up of DRα polypeptide associated with DRβ₁, DRβ₂, DRβ₃, or DRβ₄ polypeptide. The two types of structural variations comprise primary amino acid sequences which differ by as much as 35%. The class II polypeptide chains possess domains that are specific structural subunits containing variable sequences that distinguish among class II α genes and class II β genes. These allelic variation sites form antigen binding clefts, which represent individual structural differences in immune recognition. Class II molecules are expressed codominantly, but in contrast to class I, exhibit a restricted tissue distribution: they are present only on the surface of cells of the immune system. Such cells include antigen-presenting cells, for example, macrophages, dendritic cells, and Langerhans cells; epithelial tissue cells that interact with immune system, including thymic epithelial cells; B lymphocytes, monocytes and mast cells; and T cells when they are induced.

The three-dimensional structure of three different DR molecules and a DQ molecule of class II MHC has been determined (Brown et al. (1993), Nature 364: 33; Stem et al. (1994) Nature 388: 215; Ghosh et al. (1995) Nature 378: 457; Dessen et al. (1997) Immunity, 7: 473; Lee et al. (2001) Nature Immunol. 2(6): 501-507). Overall, their structure is very similar to that of class I molecules. The peptide binding site is composed of the first domains of the α and β chains, which, in contrast to class I, is open on both sides, allowing the binding of longer (12-24 residues long) peptides (Chicz et al. (1992) Nature, 358: 764). An additional binding site on the second domain of both α and β chains interacts with the CD4 molecule, expressed selectively on helper T (Th) cells. This molecule has a co-receptor function for T helper (Th) cells, analogous to that of CD8 for cytotoxic T (Tc) cells.

A peptide bound to a class II MHC molecule is presented in such a way that particular T cells are activated. There are generally two types of T cells: T helper 1 (Th1) and T helper 2 (Th2). Th1 cells participate in providing cell-mediated immunity, which is generally pro-inflammatory. When activated, Th1 cells produce pro-inflammatory cytokines such as interferon(IFN)-γ and interleukin (IL)-2. Th2 cells participate in providing humoral immunity, which is generally non-inflammatory. When activated, Th2 cells produce non-inflammatory cytokines such as IL-4, IL-5, IL-10, and IL-13. The activated T cells may also be induced to proliferate or to undergo apoptosis. Thus, peptides bound to, and presented by, MHC molecules may activate either Th1 or Th2, shifting the balance of pro-inflammatory and non-inflammatory responses, depending on the identity of the peptides.

A large body of evidence has demonstrated that susceptibility to many diseases, in particular autoimmune diseases, is strongly associated with specific alleles of the major histocompatibility complex (reviewed in Tiwari and Terasaki (1985), “HLA and disease association,” New York; Springer Verlag). Autoimmune diseases include rheumatoid arthritis (RA), multiple sclerosis (MS), human type I or insulin-dependent diabetes mellitus (IDDM), autoimmune uveitis, primary biliary cirrhosis (PBC) and celiac disease. Although a few class I-associated diseases exist, most autoimmune conditions have been found to be associated with class II alleles. MHC class II molecules are of great importance in the selection and activation of CD4+ T lymphocytes, which regulate the immune responses against protein antigens. Genomic analysis has identified specific individual allelic variants of HLA in associations with Hodgkin's disease, multiple sclerosis, rheumatoid arthritis, pemphigus vulgaris, insulin dependent diabetes mellitus (IDDM, Type I diabetes), and celiac disease, among others (Thomson (1995) Crit. Rev. Clin. Lab. Sci. 32: 183-219; Nepom and Erlich (1991) Annu. Rev. Immunol. 9: 493-525; Tiwari, above).

Type 1 diabetes (i.e., Insulin-dependent diabetes mellitus, (IDDM)) represents 20% of all human diabetes, and is the most serious form of the disease, with highest morbidity and mortality. Up to 800,000 people in the U.S. are estimated to have IDDM, with about 30,000 new cases diagnosed each year. The incidence of IDDM has been rising over the past few decades in certain regions of the US and some European countries, particularly in Finland and England. Some complications arising from long-standing diabetes are vascular disease, microvascular disease, eye complications, diabetic nephropathy, diabetic neuropathy, diabetic foot problems, and skin and mucous membrane problems.

IDDM is a progressive autoimmune disease, in which the β cells of the pancreas that produce insulin are slowly destroyed by the body's own immune system. Certain proteins, such as glutamic acid decarboxylase (GAD), insulin, and islet cell antigens, serve as autoantigens, becoming targets of self-attack of the immune system. Of these autoantigens, GAD has been suggested as a dominant autoantigen in the pathogenesis of the disease. It is unknown what triggers this cascade of aberrant immune events, but in humans, IDDM susceptibility and resistance has been associated with the HLA-DQ molecules encoded by alleles of certain HLA-DQB1 and DQA1 loci. Such HLA-DQ molecules are the combined protein products of specific HLA-DQB1 and DQA1 alleles known as DQB1*0201, DQB1*0302, DQB1*0304, DQB1*0401, DQB1*0501, DQB1*0502; and DQA1*0301, DQA1*0302, DQA1*0303, DQA1*0501. These alleles maybe encoded on one haplotype (“cis” alleles) such as DQB1*0201-DQA1*0501-DRB1*0301 and DQB1*0302-DQA1*0301-DRB1*0401. Alternatively, the alleles may be encoded on different haplotypes (“trans” alleles). An example of “trans” alleles is the combination of DQB1*0201 on DQB1*0201-DQA1*0501-DRB1*0301, or DQA1*0301 on DQB1*0301-DQA1*0301-DRB1*0404. Individuals carrying both DQB1*0201-DQA1*0501 and DQB1*0302-DQA1*03 haplotypes have the highest risk of developing IDDM. (Yu et al. (2000) Eur. J. Immunol. 30: 2497-2506). Additionally, 95% of Caucasians with IDDM carry the alleles DRB1*0301 or DRB1*0401, or both. In a mouse model system of diabetes using transgenic animals expressing Aβ°/HLA-DQ8 and HLA-DR3, naturally processed peptides derived from GAD are bound to HLA-DQ8 and/or HLA-DR3 in spleens and lymph nodes. These mice develop insulitis and GAD autoreactivity spontaneously.

Currently, treatment of IDDM requires chronic administration of insulin to control hyperglycemia. Uncontrolled hyperglycemia can further damage the insulin-producing pancreatic β cells, and in the long term, create greater insulin deficiencies. Currently, oral sulfonylureas and insulin injections are the only two therapeutic agents available in the United States for treatment of IDDM. Both agents have the potential for inducing hypoglycemia as a side effect, reducing the blood glucose concentration to dangerous levels. There is no generally applicable and consistently effective means of maintaining an essentially normal fluctuation in glucose levels in IDDM. An ideal treatment would minimize the risks of hypoglycemia while keeping the glucose levels below a target value. The drug regimen is combined with regulation of dietary intake of carbohydrates to keep glucose levels in control. However, to date, there is no cure for IDDM.

Celiac disease, also known as celiac sprue or gluten-sensitive enteropathy, is a disease that results from defective gastrointestinal absorption due to hypersensitivity to cereal grain storage proteins, including glutens or its product gliadin and glutenin, present in wheat, barley, oats, and rye. The disease is caused by CD4 T cells that recognize gliadin as dietary antigen and these cells induce a Th1-mediated chronic inflammatory response, which damages the villi, causing symptoms including diarrhea, weight loss, and steatorrhea, villous atrophy, and malabsorption. Further, celiac patients may suffer from conditions that are consequences of malabsorption and malnutrition. It may also be associated with dermatitis herpetiforms, a vesicular skin eruption, irritability, depression, muscle cramps, joint pain, fatigue, and menstrual irregularities. Celiac disease is considered to be the most common genetic disease in Europe, and an estimated one in 4,700 Americans have been diagnosed with this disease, though a study suggests that as many as 1 in every 250 Americans may have some form of this disease. Celiac disease is associated with alleles DQB1*0302 and DQB1*0201 combined with DQA1*0301 and DQA1*0501. 95% of patients carry either DQB1*0201 or DQB1*0302 (Sollid et al. (1993) Gastroenterol. 105: 910). The strong HLA association is believed to be due to the capacity of DQ molecules encoded by DQB1*0201, DQA1*0501, DQB1*0302 and DQA1*0301 to efficiently present deaminated variants of glutamine-rich peptides derived from gliadin and glutenin. The same therapeutic application may therefore be useful in this disease as in IDDM.

Of the two classes of MHC molecules, class II is the primary target for immunosuppressive intervention for the following reasons: First, MHC-II molecules activate T helper (Th) cells that are central to immunoregulation, and are responsible for most of the immunopathology in inflammatory diseases. Second, most autoimmune diseases are genetically associated with class II alleles. Third, MHC-II molecules are expressed selectively on cells of the immune system, whereas MHC-I are present on most somatic cells.

A pharmaceutical agent targeting class II MHC molecules offers several advantages over most available immunosuppressive drugs. First, it would represent a disease mechanism-based intervention, which is expected to interrupt the initial event in the pathogenic cascade. Second, it can be designed to be selective for only a few class II allotypes, leaving the remainder of the antigen presenting system available for protective responses against pathogens, and therefore causing fewer immunocompromising side effects than most immunosuppressive drugs. Third, the methods and compounds could be applied without any specific knowledge of the actual autoantigens causing the disease.

To date, methods and compositions targeting the HLA-DR subclass molecules have been described, but not those that target the HLA-DQ subclass molecules.

### SUMMARY OF THE INVENTION

The present invention provides methods and compositions for treating autoimmune diseases and other unwanted immune reactions comprising administering a copolymer that binds to one or more HLA-DQ molecules and modulates DQ-restricted T cell responses. In certain preferred embodiments, the copolymers of the invention bind to HLA-DQA1 molecules, and in even more preferably to one or more of HLA molecules encoded in the alleles DQA1*0501-DQB1*0201, DQA1*0301, DQB1*0401, and DQA1*0301-DQB1*0302.

Exemplary disorders that can be treated using the subject DQ-directed copolymers include insulin-dependent diabetes mellitus (IDDM); celiac disease; rheumatoid arthritis; steroid sensitive nephrotic syndrome; mesengial IgA nephropathy; narcolepsy; neurological multiple sclerosis; relapsive polychondritis; dermiatological disorders such as dermatitis herpetiformis, atopic dermatitis, Behcet's disease, pemphigus, psoriasis; primary Sjögren's syndrome; systemic vasculitides; erythematosus; gastrointestinal disorders such as Crohn's disease; respiratory disorders such as Sommer type hypersensitivity pneumonitis, and autoimmune thyroid disease (AITD). In even more preferred embodiments, the copolymers of the present invention bind to certain HLA-DQ molecules that predispose the carrier of such molecules to IDDM and celiac disease. Such HLA-DQ molecules are the combined protein products of specific HLA-DQB1 and DQA1 alleles known as DQB1*0201, DQB1*0302, DQB1*0304, DQB1*0401, DQB1*0501, DQB1*0502; and DQA1*0301, DQA1*0302, DQA1*0303, DQA1*0501. These alleles maybe encoded on the same haplotypes (“cis” alleles) such as DQB1*0201-DQA1*0501-DRB1*0301 and DQB1*0302-DQA1*0301-DRB1*0401. The resulting HLA molecule comprising polypeptide products of “cis” alleles are herein referred to as “cis dimer.” Alternatively, the alleles may be encoded on different haplotypes (“trans” alleles). The HLA molecule comprising polypeptide products of “trans” alleles are herein referred to as “trans” dimer. An example of “trans” alleles is the combination of DQB1*0201 on DQB1*0201-DQA1*0501-DRB1*0301 and DQA1*0301 on DQB1*0301-DQA1*0301-DRB1*0404.

### Copolymers Summary

An aspect of the invention is a copolymer composition formed by random synthesis (polymerization) of the various amino acid residues. Such composition comprises a terpolymer, which is copolymers with a random sequence of at least three different amino acid residues wherein at least one amino acid is selected from each group of:
(1) acidic or neutral polar residues (aspartic acid (D), asparagine (N), glutamic acid (E), glutamine (Q)); and
(2) hydrophobic aliphatic residues and small hydrophilic hydroxy residues (leucine (L), isoleucine (I), valine (V), serine (S), threonine (T));
(3) small aliphatic residues (alanine (A), glycine (G)).

Accordingly, the copolymer is, for example, a terpolymer comprising a group of three amino acid residues in Table 1 below.

In general, in the terpolymer compositions, the copolymers are synthesized to have a molar input ratio of the amino acid components is about 2:5:3 for relative amounts of amino acids of the first group, the second group, and the third group, respectively. Alternatively, the molar input ratio of the amino acid components is about 2:25:15 for relative amounts of amino acids of the first group, the second group, and the third group, respectively. Alternatively, the molar input ratio of the amino acid components is about 2:1:0.6 for relative amounts of the first group, the second group, and the third group, respectively.

In another embodiment, the copolymer compositions are tetrapolymers, comprising four amino acid residues, at least one amino acid residue selected from each of the above three groups. Accordingly, the copolymer of the invention is, for example, a tetrapolymer comprising a group of four amino acid residues in Table 2 below.

Preferred embodiments of the inventions are copolymer compositions comprising a random sequence of one of the following sets of amino acid residues:

aspartic acid, alanine, leucine, and glutamic acid (DALE);

aspartic acid, alanine, isoleucine, and glutamic acid (DAIE);

aspartic acid, alanine, valine, and glutamic acid (DAVE);

aspartic acid, alanine, threonine, and glutamic acid (DATE);

aspartic acid, glycine, leucine, and glutamic acid (DGLE);

aspartic acid, glycine, isoleucine, and glutamic acid (DGIE);

aspartic acid, glycine, valine, and glutamic acid (DGVE); or

aspartic acid, glycine, threonine, and glutamic acid (DGTE).

In general, these compositions are synthesized to have a molar output ratio of amino acid components, as they appear above, of about 1:10:3:1, or 1:15:3:1 respectively. Alternatively, the molar output ratio of amino acid components is about 1:25:15:5, respectively. Alternatively, the molar output ratio of amino acid components is about 1:3:1.5:0.2, respectively. Molar output ratios have a variability range of about 10% between the different amino acids. A preferred molar input ratio for the synthesis of a copolymer composition for D:A:X:E or D:G:X:E is about 1:5:3:1, wherein X is L, I, V, S, or T. Alternatively, the molar input ratio of these amino acids is about 1:25:15:5, or 1:1:1.5:0.2.

In other embodiments, the subject DQ-directed copolymers are a mixture of copolymers with randomized or partially randomized amino acid sequences containing amino acid residues wherein at least one amino acid is selected from each group of:
(1) hydrophobic aliphatic residues (such as leucine (L), isoleucine (I), valine (V), methionine (M));
(2) acidic residues (such as aspartic acid (D), glutamic acid (E));
(3) small hydrophilic residues (such as serine (S), threonine (T), cysteine (C)); and
(4) small aliphatic residues (such as alanine (A), glycine (G)).

In one embodiment, the copolymer is derived using the amino acids glutamic acid (E) and/or aspartic acid (D), leucine (L), serine (S) and alanine (A), and is referred to herein as an “ELSA” copolymer.

In certain other embodiments, the subject DQ-directed copolymers are a mixture of randomized or partially randomized amino acid sequence containing at least five different amino acid residues wherein at least one amino acid is selected from each group of:
(1) acid residues (such as aspartic acid (D), glutamic acid (E));
(2) hydrophobic aliphatic residues (such as leucine (L), isoleucine (I), valine (V), methionine (M))
(3) bulky hydrophobic residues (such as tyrosine (Y), phenylalanine (F));
(4) small hydrophilic residues (such as serine (S), cysteine (C), threonine (T)); and
(5) small aliphatic residues (such as alanine (A), glycine (G)).

Another exemplary copolymer is derived using the amino acid residues glutamic acid (E) and/or aspartic acid (D), leucine (L), tyrosine (Y) and valine (V), and is referred to herein as a “DLYV” copolymer.

In another embodiment, any of the copolymers can further comprise an additional amino acid residue, wherein the additional amino acid residue is found at a certain amino acid sequence position in an autoantigenic peptide for an autoimmune disease, such as diabetes. Such amino acid influences the affinity of the peptide for a functional binding to the class II MHC protein associated with the autoimmune disease. Such copolymer has T cell stimulatory activity when in a complex with a class II MHC protein. For example, an additional amino acid to any of the combination above is a lysine residue (K). The K residue is present in sufficient molar output ratio to increase T-cell stimulation by the copolymer complexed with a class II MHC protein. Further, the K residue may be present in sufficient molar output ratio to increase aqueous solubility of the copolymer. In another embodiment, the copolymer may contain proline (P) residues.

A certain ratio of amino acids to be incorporated into the random copolymer may be used. Preferred random copolymers of the present invention comprise amino acid residues K, E, A, S, V, and P. A preferred molar input ratio of K:E:A:S:V is 0.3:0.7:9:0.5:0.5:0.3.

In other embodiments, the copolymer amino acid sequence is not completely random, and has “anchor” residues which occur with regular spacing in the resulting polymer. Preferably, the copolymer has a general sequence:

[XXXa₁XXXXXXXXa₂XX]n,

wherein Xa₁ and Xa₂ are each an acidic amino acid residue selected from glutamic acid and aspartic acid, X is any selected amino acid residue, and 2≦n≦8.

Preferably, the copolymer can be synthesized to have one of the general sequences:

1. [XXEXXXXXXXEXX]₄

2. [XXEXXXXXXXDXX]₄

3. [XXDXXXXXXXDXX]₄

4. [XXDXXXXXXXDXX]₄

5. [XXEXXVXXXXDXX]₄

6. [XXDXXVXXXXDXX]₄

7. [XXDXXVXXXXEXX]₄

8. [XXEXXVXXXXEXX]₄

wherein X is A, S, V, K, or P.

In a preferred embodiment, the molar input ratio of A:S:V:K:P is 5:1:1:1:0.5.

The copolymer is capable of binding to a class II MHC protein, for example, a human class II MHC protein such as HLA-DQ2 encoded by alleles DQA1*0501-DQB1*0201 or HLA-DQ8 encoded by alleles DQA1*03-DQB1*0302 allele. Further, the copolymer is capable of binding to a class II MHC protein of a subject animal such as a mouse, for example, IA^{g7} protein. In preferred embodiments, the copolymers compositions of the present invention bind to one or more DQ isotypes with an average K_{d} of 1 μM or less, and more preferably an average K_{d} less than 100 nM, 10 nM or even 1 nM. Another way to identify preferred copolymers is based on competitive binding assays, such as described in Sidney et al. (2002) J. Immunol. 169: 5098, which is expressed as an IC₅₀ value. Preferred copolymers of the present invention have IC₅₀'s less than 1 μM, more preferably less than 500 nM, and even more preferably less than 100 nM.

The copolymer provided herein is at least about 30 residues in length, at least about 40 residues in length, or the copolymer is at least about 50 residues in length. Further, the copolymer is no greater than about 90 residues in length, no greater than about 80 residues in length, or no greater than about 70 residues in length. Preferably, the random copolymers are about 10 to 100 amino acid residues long, more preferably 20 to 80 amino acid residues long, 30 to 70 amino acid residues long, even more preferably 40 to 60 amino acid residues long, and most preferably about 50 amino acid residues long. When synthesized, a typical preparation of random copolymers is a mixture of peptides of various lengths, the majority of which are of the desired length but containing shorter or longer peptides inevitably created by the currently available synthetic processes.

In certain preferred embodiments, the subject copolymers are formulated for use as a medicament so as to have a polydispersity less than 25,000, and more preferably less than 10000, 5000, 1000, 500, 100, 50, or less than 10.

### Method of Treatment Summary

Another aspect of the invention is methods of treatment of an autoimmune disease comprising administering a copolymer composition that functionally binds to an HLA-DQ molecule associated with the autoimmune disease, thereby activating T cell recognition. In certain embodiments, the subject copolymers bind to autoimmune-associated HLA-DQ istotypes, such as one or more of DQB1*0201, DQB1*0302, DQB1*0304, DQB1*0401, DQB1*0501, DQB1*0502; and DQA1*0301, DQA1*0302, DQA1*0303, DQA1*0501, with a K_{d} at least 10 times less than the copolymer's K_{d} for binding HLA-DR molecules and/or other DQ isotypes.

Another aspect of the invention is methods of treatment of an unwanted immune response which are mediated by HLA-DQ molecules comprising administering a copolymer composition that functionally binds to an HLA-DQ molecule associated with such unwanted immune responses. Yet another aspect of the invention is methods of treatment of allergies and allergic reactions mediated by HLA-DQ molecules comprising administering a copolymer composition that functionally binds to an HLA-DQ molecule associated with the allergy. An aspect of the invention also provides methods of treatment of a disease treatable by administration of a copolymer composition that functionally binds to an HLA-DQ molecule associated with such a disease.

A preferred embodiment of the invention provides a method for treating a diabetic condition in a subject, comprising administering to the subject a composition comprising a copolymer having amino acids polymerized in a random sequence, the amino acids comprising at least one residue from each of the following groups:
(1) acidic or neutral polar residues (aspartic acid (D), asparagine (N), glutamic acid (E), glutamine (Q)); and
(2) hydrophobic aliphatic residues and small hydrophilic hydroxy residues (leucine (L), isoleucine (I), valine (V), serine (S), threonine (T));
(3) small aliphatic residues (alanine (A), glycine (G)).

thereby treating the subject for the diabetic condition. In general, in the copolymer, the acidic residue is glutamic acid and/or aspartic acid; the neutral residue is alanine and/or glycine; and the hydrophobic aliphatic amino acid is leucine, isoleucine, valine, and/or threonine.

The subject of the treatment can be a human. Alternatively, the subject is a non-human animal, such as a rodent, including a rat, mouse, or hamster. For example, the subject is a non-obese diabetic (NOD) mouse or a streptozoticin-induced diabetic mouse.

In another embodiment, the method of treatment is carried out using any of the copolymers of invention, preferably, a copolymer that comprises a polypeptide comprising at least one amino acid residue selected from each of the following groups:
(2) acidic residues (aspartic acid (D), glutamic acid (E));
(4) small aliphatic residues (alanine (A), glycine (G));
(1) hydrophobic aliphatic residues (leucine (L), isoleucine (I), valine (V), methionine (M)); and
(3) small hydrophilic residues (serine (S), cysteine (C), threonine (T)).

In addition, the copolymer may comprise proline (P).

In certain embodiments, the methods allow continuous treatment of autoimmune diseases by a sustained-release carrier such as transdermal patches, implantable medical devices coated with sustained-release formulations, or implantable or injectable pharmaceutical formulation suitable for sustained-release of the active components.

The methods for treatment of the present invention also provide for administration of the copolymer in combination with other drugs. The subject copolymers can be administered conjointly with other active ingredients, such as anti-inflammatory agents, growth factors, cytokines, immunosuppressant agents, or anti-hypertensive drugs, drugs to treat lipid disorders or anti-obesity drugs in diabetic patients. For example, the subject copolymers can be used in conjunction with cyclooxygenase inhibitors, and inhibitors of TNF-α; IL-1 or ICAM-1. Alternatively, the additional agent is an immune suppressive agent. The immune suppressive agent can be a drug or a protein. The drug is at least one of a rapamycin; a corticosteroid; an azathioprine; mycophenolate mofetil; a cyclosporine; a cyclophosphamide; a methotrexate; a 6-mercaptopurine; FK506; 15-deoxyspergualin; a sphingosine-1-phosphate receptor agonist such as FTY 720 (2-amino-2-(2-[4-octylphenyl]ethyl)-1,3-propanediol hydrochloride), and other phosphonate analogs (Forrest et al. (2004) JPET 309:758-768); a mitoxantrone; a 2-amino-1,3-propanediol; a 6-(3-dimethyl-aminopropionyl)forskolin; and a demethimmunomycin. The protein is at least one of hul 124; BTI-322; allotrap-HLA-B270; OKT4A; Enlimomab; ABX-CBL; OKT3; ATGAM; basiliximab; daclizumab; thymoglobulin; ISAtx247; Medi-500; Medi-507; Alefacept; efalizumab; infliximab; and an interferon.

Examples of anti-hypertensive drugs include β-blockers, cathepsin S inhibitors, and ACE inhibitors. Examples of drugs to treat lipid disorders include HMG-CoA reductase inhibitors, nicotinic acid, bile acid sequestrants, and fibric acid derivatives. Examples of anti-obesity drugs include P-3 agonists, CB-1 antagonists, appetite suppressants, such as, for example, sibutramine (Meridia), and lipase inhibitors, such as, for example, orlistat (Xenical).

In the case of treating IDDM, the copolymers of the present invention may also be conjointly administered with other known therapies for the treatment of diabetes, including PPAR agonists, sulfonylurea drugs, non-sulfonylurea secretagogues, α-glucosidase inhibitors, insulin sensitizers, insulin secretagogues, hepatic glucose output lowering compounds, and insulin. In such combination therapies, the amount of the therapeutic agent is less than prior to administering the copolymer for the same subject.

Such therapies may be administered prior to, concurrently with or following administration of the compound of the invention. Insulin includes both long and short acting forms and formulations of insulin. PPAR agonist may include agonists of any of the PPAR subunits or combinations thereof. For example, PPAR agonists may include agonists of PPAR-α, PPAR-γ, PPAR-67, or any combination of two or three of the subunits of PPAR. PPAR agonists include, for example, rosiglitazone and pioglitazone. Sulfonylurea drugs include, for example, glyburide, glimepiride, chlorpropamide, and glipizide. α-glucosidase inhibitors that may be useful in treating diabetes when administered with a copolymer of the invention include acarbose, miglitol and voglibose. Insulin sensitizers that may be useful in treating diabetes when administered with a subject copolymer include thiozolidinediones and non-thiozolidinediones. Hepatic glucose output lowering compounds that may be useful include metformin, such as Glucophage® and Glucophage® XR. Insulin secretagogues that may be useful in treating diabetes when administered with a copolymer of the invention include sulfonylurea and non-sulfonylurea drugs: GLP-1, GIP, PAC/VPAC receptor agonists, secretin, nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin.

In another embodiment of the invention, the method provides observing the frequency of diabetic episodes or the severity of diabetic episodes to gauge effectiveness of the treatment. In a related embodiment, the method of treatment provides observing a physiological parameter of the diabetic condition after administering copolymer. For example, effective treatment is monitored by measuring parameters such as decrease in free blood glucose, increase in blood insulin, increase in pancreatic insulin, increase in pancreatic mass, or increase in number of beta islet cells.

In certain embodiments, the copolymer is administered to a patient by injection, such as intravenous, subcutaneous, intramuscular, or intraperitoneal injection, or by intravenous infusion (or drip). Alternatively, the copolymer is administered by oral, transdermal, pulmonary or intraperitoneal administration.

The present invention also provides methods to prophylactically treat subjects that are at risk of developing autoimmune diseases, unwanted immune response, allergies, or any disease treatable by administering a copolymer composition, comprising administering the copolymer, so as to prevent or delay the onset of such diseases or conditions.

Another embodiment of the invention provides a kit for treating a diabetic subject comprising a copolymer having a random sequence of amino acids according to any of the amino acid copolymer compositions herein, and a container. The kit can further comprise instructions for use. The kit can provide the copolymer in a unit dose.

### Pharmaceutical Compositions Summary

Another aspect of the present invention provides a pharmaceutical composition comprising a copolymer of the present invention. The composition in some embodiments further comprises a pharmaceutically acceptable carrier and/or excipient. In certain embodiments, the pharmaceutical composition comprises one or more therapeutically effective copolymers that bind to HLA-DQ molecules, and a pharmaceutically acceptable carrier. The pharmaceutical composition may be formulated for various routes of administration, including oral, intravenous, intramuscular, subcutaneous, transdermal, pulmonary or intraperitoneal administration. In another embodiment, the pharmaceutical composition is suitable for sustained release of the active ingredients, the composition comprising biologically compatible polymers or matrices that allow slow release of the therapeutically active copolymers. Such sustained release formulations may be in a form of, for example, transdermal patches, implants, or suppositories.

In certain embodiments, the pharmaceutical composition further comprises other pharmaceutically active components, for co-administering an additional drug or agent conjointly with a copolymer as described above. The additional agent may be other copolymers, such as copolymers that cause HLA-DR mediated activation of T cells. Exemplary DR-directed copolymers include Copaxone® (glatiramer acetate, such as described in U.S. Pat. Nos. 3,849,550 and 6,214,791), YFAK and other copolymers described in PCT publication WO03/029276, and terpolymers described in PCT publication WO00/05250.

Another embodiment of the invention provides a method of manufacture of a medicament for treatment of an autoimmune disease such as diabetes or celiac disease; unwanted immune response; allergy; or any disease treatable by administering a copolymer of the present invention, comprising formulating any of the copolymers described herein, for administering to a subject in need of such treatment.

The composition may be provided in a unit dose effective for treatment of an autoimmune disease, an unwanted immune response, allergy, or any disease treatable by administering a copolymer of the present invention. The autoimmune response may be celiac disease or a diabetic condition, which may be pre-diabetes; insulin-dependent diabetes mellitus (IDDM, type I diabetes), or type II diabetes. The subject can be a human. Alternatively, the subject is a non-human animal, such as a rodent, such as a rat, mouse, or hamster. For example, the subject is a non-diabetic obese (NOD) mouse or a streptozoticin-induced diabetic mouse. The unit dose is in an amount appropriate for the body size of the subject.

### Method of Screening Summary

Another aspect of the present invention provides methods to screen for and identify copolymers that bind to HLA-DQ molecules and prevent autoimmune responses. Such methods allow identifying copolymers that are effective for treating autoimmune diseases.

In certain embodiments, the subject DQ-directed copolymers are modified, or labeled, with a moiety that facilitates the detection of the copolymers. In a preferred embodiment, the copolymers are biotinylated. In another preferred embodiment, the copolymers are modified with FITC. Exemplary copolymers are random copolymers as described above, modified with biotin or FITC. In other embodiments, the copolymers with “anchor” residues which occur with regular spacing in the resulting polymer are modified with biotin or FITC. Preferably, modified copolymers can be synthesized to have one of the general formulae:

9. Biotin-spacer-[XXEXXXXXXXEXX]n

10. Biotin-spacer-[XXEXXXXXXXDXX]n

11. Biotin-spacer-[XXDXXXXXXXDXX]n

12. Biotin-spacer-[XXDXXXXXXXEXX]n

13. Biotin-spacer-[XXEXXVXXXXDXX]n

14. Biotin-spacer-[XXDXXVXXXXDXX]n

15. Biotin-spacer-[XXDXXVXXXXEXX]n

16. Biotin-spacer-[XXEXXVXXXXEXX]n

wherein A, S, V, K, or P, the molar input ratio of which are 5:1:1:1:0.5, 2≦n≦8, and the spacer comprises two to 6 amino acid residues, preferably with the amino acid sequence SGSG. In a preferred embodiment, n=4.

These modified copolymers are used in assays and diagnostics, for example in enzyme-linked immunosorbent assay (ELISA). The labeled copolymers can also be used to determine the best sequence or preferred sequence among the copolymers that bind to an HLA molecule. Additionally, the labeled copolymer can be used in screening for other compounds not related to copolymers of the present invention that bind to or associate with HLA-DQ molecules.

The methods of screening can be used for in vivo assay in non-human animals such as a rodent, such as a rat, mouse, or hamster.

### BRIEF DESCRIPTIONS OF THE DRAWINGS

FIG. 1 shows the results obtained from a competition assay of semi-random copolymers RSP-001, RSP-002, and RSP-003 binding to HLA-DQ8 in competition with RSP-006 (biotinylated RSP-003), with data calculated as extent of competition from amount of observed complexes remaining shown on the ordinate, less background negative control, as a function of increased competitor shown on the abscissa. Afu as indicated on the ordinate means arbitrary fluorescence unit.

FIG. 2 shows the results of a competition assay of random copolymers RSP-008 (DAVE), RSP-009 (DATE), and RSP-010 (DALE) binding to HLA-DQ8 in competition with RSP-006.

FIG. 3 shows the results of a competition assay of random copolymer CO-14 (YFAK) binding to HLA-DQ8 in competition with RSP-006.

FIG. 4 shows the results of a direct binding assay of biotinylated random copolymers RSP-004, RSP-005, and RSP-006 to HLA-DQ8.

FIG. 5 shows the results of a direct binding assay of biotinylated random copolymers RSP-004, RSP-005, and RSP-006 to HLA-DR2.

FIG. 6 shows the results of a competition assay of RSP-008 (DAVE), RSP-009 (DATE), and RSP-010 (DALE) to HLA-DR2 in competition with biotinylated CLIP (class II-associated invariant chain peptide).

FIG. 7 shows RSP-001's capability to immunize mice as demonstrated by the T cell response to the copolymer after immunization.

FIG. 8 shows RSP-002's capability to immunize mice demonstrated by the T cell response to the copolymer after immunization.

FIG. 9 shows RSP-003's capability to immunize mice demonstrated by the T cell response to the copolymer after immunization.

FIG. 10 shows RSP-010 (DALE) immunize mice demonstrated by the T cell response to the copolymer after immunization.

### DETAILED DESCRIPTION OF THE INVENTION

### I. Overview

There are several autoimmune diseases that exhibit strong associations with certain alleles of human leukocyte antigen (HLA). In particular, certain diseases are associated with the HLA-DQ subclass of the alleles, either alone or in combination with the HLA-DR subclass. These diseases include IDDM and celiac disease. It is possible to identify individuals at risk of developing the diseases based on the identification of MHC class II alleles that confer susceptibility.

Random synthetic copolymers can be used to treat autoimmune diseases that are associated with HLA-DQ gene products by competing with candidate autoantigens for binding to these protein receptor molecules, or by inducing T cell anergy or even T cell apoptosis, or by suppression of T cells, such that subsequent T cell response to an autoantigen is inhibited in vivo. Further, synthetic copolymers having one or more additional components, such as amino acid analogs or derivatives added in varying quantities into the polymerization reaction, can be effective inhibitors of a variety of autoimmune T cell responses. See PCT/US02/31399 by Strominger et al., and Fridkis-Hareli et al. (2002) J. Clin. Invest. 109: 1635-1643, the entire contents of both of which are hereby incorporated herein by reference.

A major goal in the treatment of autoimmune diseases has been development of antigen-specific immunomodulating therapies that interfere with the trimolecular interaction of the autoreactive T cell receptor (TCR) with the autoantigenic peptides presented by self MHC receptors at the surface of antigen-presenting cells. These immunotherapies of T cell-mediated autoimmune diseases have been successful in animal models with known target antigens (see, for example, Weiner (1997) Immunol. Today 18:335-343; Nicholson et al. (1997) Proc. Natl. Acad. Sci. USA 94:9279-9284). The use of altered peptide ligands (APL) has been used both to treat EAE (Nicholson et al. (1997) Proc. Natl. Acad. Sci. USA 94:9279-9284; Brocke et al. (1996) Nature 379:343-346) and recently to treat MS (Bielekova et al. (2000) Nat. Med. 10:1167-1175; Kappos et al. (2000) Nat. Med. 10:1176-1182), with contradictory findings.

Insulin-dependent diabetes mellitus (IDDM), or Type I diabetes, is a serious health problem. Genetically susceptible individuals who possess certain HLA-DR and HLA-DQ subclass alleles may be monitored for autoantibodies to islet antigens which indicate onset of the disease. Treatment of such individuals at the onset of the disease to suppress autoimmune response and therefore any further destruction of the tissue is expected to be efficacious.

Another potential disease application is treatment of celiac disease, which is strongly associated with HLA encoded by alleles DQA1*0501-DQB1*0201 and -DQA1*03-DQB1*0302. Suppression of autoimmune response is expected to be efficacious in alleviating the symptoms of celiac disease.

T lymphocytes are able to recognize foreign antigen via their T cell receptor (TCR). The TCR binds to a major histocompatibility protein (MHC), which is a membrane bound glycoprotein on the cell surface of specialized antigen presenting cells. A MHC forms a complex with short, intracellularly processed peptides derived from self or foreign proteins. There are two major classes of MHC proteins, class I and class II. Class I molecules are complexed with such processed peptides derived from self or foreign proteins inside the cell, while class II molecules are in complex with those from outside the cell. Such peptide binds non-covalently to a MHC at its peptide binding groove, with a binding affinity (K_{d}) in the range of 10⁻⁶ M. The peptide binding groove of a class II MHC is open on either end and thus able to accommodate peptides of lengths ranging from 9 to 75 amino acid residues.

Glatiramer acetate, also known as Copaxone®, copolymer-1, Cop1, YEAK or GLAT, is a random amino acid copolymer composed of tyrosine (Y), glutamic acid (E), alanine (A), and lysine (K) in a molar ratio of approximately 1:1.5:5:3. Glatiramer acetate is synthesized in solution using N-carboxyamino acid anhydrides (Teitelbaum et al. (1971) Eur. J. Immunol. 1:242-248). It has been successfully developed and approved as a treatment for multiple sclerosis (MS), particularly, relapsing forms of MS (Bornstein et al. (1987) New Engl. J. Med. 317: 408-414; Johnson et al. (1995) Neurol. 45: 1268-1276), and currently is in wide use. Initially, glatiramer acetate and other related random copolymers were used to define the genetic bases of immune responsiveness, now known as class II MHC genes (McDevitt and Sela (1965) J. Exp. Med. 122: 517-532; McDevitt and Sela (1967) J. Exp. Med. 126: 969-978). Glatiramer acetate was found to be effective in suppression of experimental allergic encephalomyelitis (Teitelbaum et al. (1971) Eur. J. Immunol. 1: 242-248; Teitelbaum et al. (1973) Eur. J. Immunol. 3: 273-279; Teitelbaum et al. (1974) Clin. Immunol. Immunopathol. 3: 256-262; Aharoni et al. (1993) Eur. J. Immunol. 23: 17-25).

Although there is not a complete understanding of the mechanism of action of glatiramer acetate, it is likely that a pre-requisite for its biological activity involves an ability to bind human MHC class II molecules. The MHC allele most commonly associated with MS is HLA-DR2 (DRB1*1501), and glatiramer acetate has been shown to bind to this MHC class II molecule and activate a significant proportion (typically 15-20%) of an individual's T cells. Activation of T cells by glatiramer acetate is restricted to HLA-DR molecules, and little response is generated through the HLA-DQ molecules. (Brenner et al. (2001) J. Neuroimmunol. 115: 152-160, Fridkis-Hareli et al. (1994) Proc. Natl. Acad. Sci. USA 91: 4812-4876, Table 1). Therefore, while glatiramer acetate is effective in reducing the MS relapse rate, it does not treat other autoimmune diseases that involve HLA-DQ molecules, such as diabetes or celiac disease.

Peptide binding to class II molecules requires the presence of defined side chains at anchor positions, which all together form a particular binding motif. These anchor positions have been determined as amino acid position 1 to position 9 (or P1 to P9). The most important contacts for a peptide to make for optimal class II binding are P1, P4, P7, and P9. For the class II MHC proteins associated with diabetes, the most important positions of a peptide for interaction with protein pockets are P1 and P9. The P1 and P9 pockets are considered to be “promiscuous” in that these are large pockets that can accommodate a variety of different amino acid side chains. Using the compositions provided herein, it is found that binding is particularly tight when P1 and P9 is occupied by amino acid residue glutamic acid (E) or aspartic acid (D). Peptides with amino acid residues having distinguishing features of residues at specific positions bind to MHC molecules with predictable affinity. However, experiments using HLA-DR molecules show that, at non-anchor positions, a variation of side chains is permitted without influence on binding (Hammer et al., (1993, 1994, and 1995), above). This binding mechanism enables the presentation of many different peptides by a given allotype of HLA. The side chains at anchor positions interact with specific pockets within the binding site, whereas those at non-anchor positions point outward, and are available for recognition by T cell receptors (TCR) on Th cells.

It is therefore conceivable that a compound with the same binding motif as autoantigenic peptides but with different residues at non-anchor positions would bind to the disease associated MHC molecules, thereby preventing the activation of autoimmune T cells, and thus interrupting the disease process. According to this model, a copolymer having the types of amino acid residues that most closely fit pockets in the protein at key positions or “anchor” residues would be most effective in ameliorating symptoms of the autoimmune disease. The mechanism whereby such a compound would exert its effect is competitive antagonism for the antigen-presenting site. Compounds binding selectively to class II molecules involved in a particular autoimmune disease are therefore expected to interfere specifically with that disease. Additional peptides which bind to MHC molecules and inhibit T cell activation have been disclosed in, for example, International Patent applications WO 92/02543, WO 93/05011, WO 95/07707.

Alternatively, a compound that replaces an autoantigenic peptide may activate a different set of T cells than the autoantigenic peptide would (Vignali and Strominger (1994) J. Exp. Med. 179: 1945-1956). In instances where the autoimmune response is characterized by undesirable inflammatory responses mediated by Th1 cells, activation of Th2 cells to produce or increase production of an immunosuppressive cytokine IL-10 instead of Th1 cells may alleviate the symptoms of the autoimmune reaction and result in suppression of undesired immune response. It is likely that the important positions for interaction of class II MHC protein complexes with T cells are P2, P4, and P5. See Wucherpfennig et al. (1994) J. Exp. Med. 179: 279; see also Bettelli et al. (1998) J. Immunol. 161: 3299; and Aharoni et al. (1998) J. Neuroimmunol. 91: 135, showing stimulation of T cells with mouse class II MHC protein complexes, and Duda et al. (2000) J. Cell Immunol. 105: 967, with human class II MHC protein complexes. Arnon et al. (2003) Proc. Nat. Acad. Sci. USA 100(24):14157-62, showed that treatment with Cop I induces specific Th2 cells in the central nervous system of mice, and that these Th2 cells secrete immunosuppressive cytokines.

Recently the crystal structure of a complex of a human insulin peptide and HLA encoded by alleles DQA1*03-DQB1*0302 has been determined. (Lee et al. (2001) Nature Immunol. 2: 501-507) Based on this structure, peptide binding studies with HLA encoded by alleles DQA1*03-DQB1*0302 (Yu et al. (2000) Eur. J. Immunol. 30: 2497-2506), and experimental results, provided herein below are copolymers that would be able to bind to certain HLA molecules.

Without being limited by any specific mechanism of action, a first group of amino acids was chosen herein that when incorporated into a copolymer will occupy P1 and P9 pockets. The first group of amino acids was chosen on the basis of a number of different criteria, for example, analysis of data shown herein in Table 4, and includes aspartic acid (D), glutamic acid (E), asparagine (N) and glutamine (Q). A second group of amino acids are chosen to interact with the TCR when occupying the P4 position, which can also be promiscuous. See Herman et al. (1999) J. Immunol. 163: 6275. The second group of amino acids is valine (V), isoleucine (I), leucine (L), serine (S) and thieonine (T). Additional amino acids may be used, such as lysine (K), that affect the charge of the copolymer, and therefore presumably the aqueous solubility, and in addition may when occupying a position in the copolymer, interact with the TCR to alter the response of a T cell.

### II. Definitions

The term “allotype” means a distinct antigenic form of a serum protein that results from allelic variations present on the immunoglobulin heavy chain constant region.

The term “anergy” means unresponsiveness of the immune system of a subject, either on the cellular level or on the organismic level, to an antigen.

The term “associated with” means “coexistent with” or “in correlation with.” The term does not necessarily indicate causal relationship, though such relationship may exist.

The term “autoimmune condition” or “autoimmune disease” means a disease state caused by an inappropriate immune response that is directed to a self-encoded entity which is known as an autoantigen. An autoimmune disease is a class of disorder which include Hashimoto's thyroiditis; idiopathic myxedema, a severe hypothyroidism; multiple sclerosis, a demyelinating disease marked by patches or hardened tissue in the brain or the spinal cord; myasthenia gravis which is a disease having progressive weakness of muscles caused by autoimmune attack on acetylcholine receptors at neuromuscular junctions; Guillain-Barre syndrome, a polyneuritis; systemic lupus erythematosis; uveitis; autoimmune oophoritis; chronic immune thrombocytopenic purpura; colitis; diabetes; celiac disease which is gluten intolerance; Grave's disease, which is a form of hypothyroidism; psoriasis; pemphigus vulgaris; and rheumatoid arthritis (RA).

The term “binding” refers to a direct association between two molecules, due to, for example, covalent, electrostatic, hydrophobic, ionic, and/or hydrogen-bond interactions under physiological conditions, and including interactions such as salt bridges and water bridges.

The term “cis” refers to two alleles encoded by gene loci on the same haplotype while “trans” refers to two alleles encoded by genes on two different haplotypes. When two polypeptides that form an HLA protein are from cis alleles, the product is herein referred to as “cis dimer.” When two polypeptides that form an HLA protein are from trans alleles, the product is herein referred to as “trans dimer.”

The term “copolymer” means a polymer of amino acids having a random amino acid sequence comprising a plurality of amino acid residues of different kinds. Amino acid residues may be naturally occurring or synthetic analogs. Copolymers also include derivatives, including chemically modified polypeptides and peptidomimetics, and may include chemical bonds other than naturally occurring peptide bonds.

The term “diabetes” as used herein means any manifested symptoms of diabetes in any mammal including experimental animal models, and including human forms such as insulin-dependent diabetes mellitus (IDDM, type I diabetes), that is linked genetically to alleles DQA1*0501-DQB1*0201 (alleles for HLA-DQ2) or DQA1*03-DQB1*0302 (alleles for DQ8), type II diabetes, early stage diabetes, and a pre-diabetic condition characterized by mildly decreased insulin or mildly elevated blood glucose levels. While the current diabetes epidemic is primarily type II or adult onset diabetes and is characterized as insulin resistance, the disease may manifest as damage to beta cells and insulin insufficiency. A “pre-diabetic condition” describes a condition in a mammal not formally diagnosed with diabetes, but is suspected of having a diabetic or related condition by, for example, demonstrating a symptom in terms of insulin or glucose level and having susceptibility to diabetes or a related condition due to family history, genetic predisposition, or obesity in the case of type II diabetes, or when a mammal is subject to risk of recurrence of diabetes when it has previously had diabetes or a related condition.

The term “haplotype” is defined as a contiguous region of genomic DNA resulting from a non-random distribution of alleles on several gene loci of a same chromosome due to a low inter-chromosomal recombination in this particular region of the genome. As the MHC genes are proximal to each other on the chromosome, genetic recombination rarely occurs within the MHC and most individuals will inherit an intact set of parental alleles from each parent; such a set of linked genes is referred to as a haplotype, the MHC genes found in one haploid genome.

The term “heterologous cell” means a cell for production of an MHC protein which is unrelated to a cell of a subject, e.g., the heterologous cell is not a cell of a mammal. The heterologous cell for example can be from a cold blooded animal, for example, from an invertebrate; the heterologous cell is an insect cell, or a cell of a microorganism such as a yeast cell.

The term “HLA molecule” means any class II major histocompatibility complex glycoproteins. The term “HLA-DQ molecule” or “HLA-DR molecule” each refers to any one of HLA-DQ subtypes or HLA-DR subtypes.

The term “IC₅₀” means the concentration of an agent that produces a 50% reduction in the effect compared to that in the absence of the agent being tested for IC₅₀

The term “molar input ratio” means the molar ratio of amino acids used to prepare a random copolymer. The input molar ratio determines how much of each amino acid is used to synthesize a random copolymer.

The term “molar output ratio” means the molar ratio of the amino acids that comprise a random copolymer composition. The output molar ratio can be determined by amino acid composition analysis of a random copolymer composition sample. In general, smaller amino acids are more efficiently incorporated into a polypeptide, resulting in a higher output ratio of the amino acid in comparison to other amino acid components than indicated by the input molar ratio.

The term “MHC activity” refers to the ability of an MHC molecule to stimulate an immune response, e.g., by activating T cells. An inhibitor of MHC activity is capable of suppressing this activity, and thus inhibits the activation of T cells by MHC. In preferred embodiments, a subject inhibitor selectively inhibits activation by a particular class II MHC isotype or allotype. Such inhibitors may be capable of suppressing a particular undesirable MHC activity without interfering with all MHC activity in an organism, thereby selectively treating an unwanted immune response in an animal, such as a mammal, preferably a human, without compromising the animal's immune response in general.

The term “antigen binding groove” or “peptide binding groove” refers to a three dimensional antigen interactive site on the surface of the Class II MHC protein molecule (Stem et al. (1994) Nature 368: 215) that is formed by surfaces of both the α and β subunits of the Class II MHC protein molecule. The term “surface of a class II MHC HLA protein” includes the portion of the protein molecule in its three-dimensional configuration which is in contact with its external environment, including those features of the protein that interact with aqueous solvent and are capable of binding to other cell components such as nucleic acids, other proteins, and peptides.

Terms “P1 pocket” and “P4 pocket” include three dimensional polymorphic regions on the peptide binding surface of the Class II MHC protein molecule that accommodate amino acid residue side chains from a peptide that is bound to the class II MHC protein (Fridkis-Hareli et al (1998) J. Immunol. 160:4386-4397; Fridkis-Hareli et al. (2000) Human Immunol. 61:640; Fridkis-Hareli et al. (2001) Human Immunol. 62:753-763), including a bound naturally occurring antigen or epitope, and a bound synthetic peptide or copolymer.

The terms “P-1 position” and “P5 position” refer to amino acid residues on the Class II MHC protein molecule peptide complex which directly contact the T-cell receptor (Fridkis-Hareli et al. (2000) Human Immunol. 61:640; Fridkis-Hareli et al. (2001) Human Immunol. 62:753-763). The P-1 position refers to the amino acid which precedes the amino acid residue of the peptide that occupies the P1 pocket. The P5 position refers to the amino acid residue that follows the amino acid residue that occupies the P4 pocket in amino acid sequence of a peptide or polypeptide. The P2, P3, and P5 residues are TCR contact residues. Similarly, the P9 position refers to the amino acid residue located four positions beyond the P5 position in amino acid sequence of a peptide or polypeptide.

The term “patient” refers to an animal, preferably a mammal, including humans as well as livestock and other veterinary subjects.

The terms “peptide”, “polypeptide”, and “protein” are used interchangeably herein. These terms refer to unmodified amino acid chains, and also include minor modifications, such as phosphorylations, glycosylations and lipid modifications. The terms “peptide” and “peptidomimetic” are not mutually exclusive and include substantial overlap.

A “peptidomimetic” includes any modified form of an amino acid chain, such as a phosphorylation, capping, fatty acid modification, and including unnatural backbone and/or side chain structures. As described below, a peptidomimetic comprises the structural continuum between an amino acid chain and a non-peptide small molecule. Peptidomimetics generally retain a recognizable peptide-like polymer unit structure. Thus, a peptidomimetic may retain the function of binding to a HLA protein forming a complex which activates autoreactive T cells in a patient suffering from an autoimmune disease.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11: 1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine (G), alanine (A), valine (V), cysteine (C), leucine (L), isoleucine, serine (S), threonine (T), methionine (M), glutamic acid (E), aspartic acid (D), glutamine (Q), asparagine (N), lysine (K), arginine (R), proline (P), histidine (H), phenylalanine (F), tyrosine (Y), and tryptophan (W). Most of the amino acids used in the copolymers of the present invention may exist in particular geometric or stereoisomeric forms. In preferred embodiments, the amino acids used to form the subject copolymers are (L)-isomers, although (D)-isomers may be included in the copolymers such as at non-anchor positions or in the case of peptidomimetic versions of the copolymers. As used herein, “amino acid” can include one or more components which are amino acid derivatives and/or amino acid analogs as defined herein. For example, in a copolymer compositing having “tyrosine” residues, a portion of one or more of those residues can be substituted with homotyrosine.

The term “amino acid residue” further includes analogs, derivatives, and congeners of any specific amino acid referred to herein, as well as C-terminal or N-terminal protected amino acid derivatives. The term “derivative” of an amino acid means a chemically related form of that amino acid having an additional substituent, for example, N-carboxyanhydride group, a γ-benzyl group, an ∈,N-trifluoroacetyl group, a halide group attached to an atom of the amino acid, or the amino acid may be modified with an N-terminal or C-terminal protecting group.

The term “amino acid analog” means a chemically related form of that amino acid having a different configuration, for example, an isomer, or an organic molecule with the approximate size, charge, and shape of the amino acid. For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups. For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention. In general, “amino acid” herein includes variations of natural amino acids, including amino acids in a polypeptide form with one or more non-peptide or peptidomimetic bonds between two adjacent residues.

The term “hydrophobic” amino acid means aliphatic amino acids alanine (A), glycine (G), isoleucine (I), leucine (L), proline (P), and valine (V), the terms in parentheses being the one letter standard code abbreviations for each amino acid, and aromatic amino acids tryptophan (W), phenylalanine (F), and tyrosine (Y). These amino acids confer hydrophobicity as a function of the length of aliphatic and size of aromatic side chains, when found as residues within a protein or a peptide.

The term “hydrophilic hydroxy” amino acid means serine (S) or threonine (T).

The term “charged” amino acid means amino acids aspartic acid (D), glutamic acid (E), histidine (H), arginine (R) and lysine (K), which confer a positive (H, K, and R) or negative (D, E) charge at physiological values of pH in aqueous solutions on peptides or proteins containing these residues. Histidine (H) is hydrophobic at pH 7, and charged at pH 6.

“Prevent”, as used herein, means to delay or preclude the onset of, for example, one or more symptoms, of a disorder or condition.

The term “prodrug” is intended to encompass compounds that, under physiological conditions, are converted into the inhibitor agents of the present invention. A common method for making a prodrug is to select moieties which are hydrolyzed under physiological conditions to provide the desired biologically active drug. In other embodiments, the prodrug is converted by an enzymatic activity of the patient or alternatively of a target pathogen.

“Treat”, as used herein, means at least lessening the severity or ameliorating the effects of, for example, one or more symptoms, of a disorder or condition.

The term “ED₅₀” means the dose of a drug that produces 50% of its maximum response or effect. Alternatively, it may refer to the dose that produces a pre-determined response in 50% of test subjects or preparations.

The term “LD₅₀” means the dose of a drug that is lethal in 50% of test subjects.

The term “therapeutic index” refers to the therapeutic index of a drug defined as LD₅₀/ED₅₀.

The terms “structure-activity relationship” or “SAR” refer to the way in which altering the molecular structure of drugs alters their interaction with a receptor, enzyme, etc.

The term “aliphatic” refers to a linear, branched, cyclic alkane, alkene, or alkyne. In certain embodiments, aliphatic groups in the present invention are linear or branched and have from 1 to about 20 carbon atoms.

The term “alkyl” refers to the radical of saturated aliphatic groups, including straight-chain alkyl groups, branched-chain alkyl groups, cycloalkyl(alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer carbon atoms. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN, and the like.

The term “heteroatom” refers to an atom of any element other than carbon or hydrogen. Illustrative heteroatoms include boron, nitrogen, oxygen, phosphorus, sulfur and selenium, and alternatively oxygen, nitrogen or sulfur.

The term “aryl” includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfliydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

### III. Exemplary Embodiments

### Copolymers

The present invention provides compounds that bind to and activate T cells in an HLA-DQ-mediated manner in addition to, or instead of, an HLA-DR-mediated manner. The present invention also provides compounds that bind to class II MHC molecules and prevent auto-antigenic peptides from activating T cells in an HLA-DQ-mediated manner.

An aspect of the invention a copolymer composition formed by random synthesis (polymerization) of the various amino acid residues. Such composition comprises a terpolymer, which is copolymers with a random sequence of at least three different amino acid residues wherein at least one amino acid is selected from each group of:
(1) acidic or neutral residues (aspartic acid (D), asparagine (N), glutamic acid (E), glutamine (Q)); and
(2) hydrophobic aliphatic residues and small hydrophilic hydroxy residues (leucine (L), isoleucine (I), valine (V), serine (S), threonine (T));
(3) small aliphatic residues (alanine (A), glycine (G)).

Accordingly, the copolymer is, for example, a terpolymer comprising three amino acid residues selected from the groups of three amino acid residues in Table 1:

TABLE 1
Terpolymer compositions

| | | | |
|---|---|---|---|
| aspartic acid: | alanine: | leucine | (DAL) |
| aspartic acid: | alanine: | isoleucine | (DAI) |
| aspartic acid: | alanine: | valine | (DAV) |
| aspartic acid: | alanine: | threonine | (DAT) |
| aspartic acid: | alanine: | serine | (DAS) |
| asparagine: | alanine: | leucine | (NAL) |
| asparagine: | alanine: | isoleucine | (NAI) |
| asparagine: | alanine: | valine | (NAV) |
| asparagine: | alanine: | threonine | (NAT) |
| asparagine: | alanine: | serine | (NAS) |
| glutamic acid: | alanine: | leucine | (EAL) |
| glutamic acid: | alanine: | isoleucine | (EAI) |
| glutamic acid: | alanine: | valine | (EAV) |
| glutamic acid: | alanine: | threonine | (EAT) |
| glutamic acid: | alanine: | serine | (EAS) |
| glutamine: | alanine: | leucine | (QAL) |
| glutamine: | alanine: | isoleucine | (QAI) |
| glutamine: | alanine: | valine | (QAV) |
| glutamine: | alanine: | threonine | (QAT) |
| glutamine: | alanine: | serine | (QAS) |
| aspartic acid: | glycine: | isoleucine | (DGI) |
| aspartic acid: | glycine: | valine | (DGV) |
| aspartic acid: | glycine: | threonine | (DGT) |
| aspartic acid: | glycine: | serine | (DGS) |
| asparagine: | glycine: | isoleucine | (NGI) |
| asparagine: | glycine: | valine | (NGV) |
| asparagine: | glycine: | threonine | (NGT) |
| asparagine: | glycine: | serine | (NGS) |
| glutamic acid: | glycine: | leucine | (EGL) |
| glutamic acid: | glycine: | isoleucine | (EGI) |
| glutamic acid: | glycine: | valine | (EGV) |
| glutamic acid: | glycine: | threonine | (EGT) |
| glutamic acid: | glycine: | serine | (EGS) |
| glutamine: | glycine: | leucine | (QGL) |
| glutamine: | glycine: | isoleucine | (QGI) |
| glutamine: | glycine: | valine | (QGV) |
| glutamine: | glycine: | threonine | (QGT) |
| glutamine: | glycine: | serine | (QGS) |

In general, in the terpolymer compositions, the copolymers are synthesized to have a molar input ratio of the amino acid components is about 2:5:3 for relative amounts of amino acids of the first group, the second group, and the third group, respectively. Alternatively, the molar input ratio of the amino acid components is about 2:25:15 for relative amounts of amino acids of the first group, the second group, and the third group, respectively. Alternatively, the molar input ratio of the amino acid components is about 2:1:0.6 for relative amounts of the first group, the second group, and the third group, respectively.

In certain embodiments, the subject DQ-directed copolymers are tetrapolymers or pentapolymers, each of which copolymer is a mixture of randomized or partially randomized amino acid sequence containing at least four different amino acid residues wherein at least one amino acid is selected from each group of:
(1) hydrophobic aliphatic residues (leucine (L), isoleucine (I), valine (V), methionine (M));
(2) acidic residues (aspartic acid (D), glutamic acid (E));
(3) small hydrophilic residues (serine (S), cysteine (C), threonine (T)); and
(4) small aliphatic residues (alanine (A), glycine (G)).

Additionally, the copolymer may contain proline (P) residues. The acidic amino acid side chain serves as a key anchor residue for the P9 pocket of HLA encoded by alleles DQA1*03-DQB1*0302 and HLA encoded by alleles DQA1*0501-DQB1*0201, based on the β57 polymorphism that is linked to disease susceptibility. The aliphatic side chain serves as a good anchor for the second relevant pocket, P4. The remaining pockets are best suited to accommodate small, neutral, or hydrophobic residues. Therefore, in one embodiment, a copolymer that binds to an HLA-DQ molecule comprises a plurality of amino acid residues selected from the above-described four groups.

In one embodiment, the copolymer is derived using the amino acids glutamic acid (E) and/or aspartic acid (D), leucine (L), serine (S) and alanine (A), and is referred to herein as an “ELSA” copolymer.

In certain other embodiments, the subject DQ-directed copolymers are a mixture of randomized or partially randomized amino acid sequence containing at least four different amino acid residues wherein at least one amino acid is selected from each group of:
(1) hydrophobic aliphatic residues (such as leucine (L), isoleucine (I), valine (V), methionine (M)) and bulky hydrophobic residues (such as tyrosine (Y), phenylalanine (F), leucine (L), methionine (M));
(2) acid residues (such as aspartic acid (D), glutamic acid (E));
(3) small hydrophilic residues (such as serine (S), cysteine (C), threonine (T)); and
(4) small aliphatic residues (such as alanine (A), glycine (G)).

Additionally, the copolymer may contain proline (P) residues. An exemplary copolymer is derived using the amino acid residues glutamic acid (E) and/or aspartic acid (D), leucine (L), tyrosine (Y) and valine (V), and is referred to herein as an “DLYV” copolymer.

In an alternative embodiment, the copolymer is a tetrapolymer comprising combination of four amino acid residues, such combination selected from the groups of four amino acid residues in Table 2:

TABLE 2
Tetrapolymer compositions

| | | | | |
|---|---|---|---|---|
| aspartic acid: | alanine: | leucine: | glutamic acid | (DALE) |
| aspartic acid: | alanine: | leucine: | glutamine | (DALQ) |
| aspartic acid: | alanine: | isoleucine: | glutamic acid | (DAIE) |
| aspartic acid: | alanine: | isoleucine: | glutamine | (DAIQ) |
| aspartic acid: | alanine: | valine: | glutamic acid | (DAVE) |
| aspartic acid: | alanine: | valine: | glutamine | (DAVQ) |
| aspartic acid: | alanine: | threonine: | glutamic acid | (DATE) |
| aspartic acid: | alanine: | threonine: | glutamine | (DATQ) |
| aspartic acid: | alanine: | serine: | glutamic acid | (DASE) |
| aspartic acid: | alanine: | serine: | glutamine | (DASQ) |
| asparagine: | alanine: | leucine: | glutamic acid | (NALE) |
| asparagine: | alanine: | isoleucine: | glutamic acid | (NAIE) |
| asparagine: | alanine: | valine: | glutamic acid | (NAVE) |
| asparagine: | alanine: | threonine: | glutamic acid | (NATE) |
| asparagine: | alanine: | serine: | glutamic acid | (NASE) |
| aspartic acid: | glycine: | leucine: | glutamic acid | (DGLE) |
| aspartic acid: | glycine: | leucine: | glutamine | (DGLQ) |
| aspartic acid: | glycine: | isoleucine: | glutamic acid | (DGIE) |
| aspartic acid: | glycine: | isoleucine: | glutamine | (DGIQ) |
| aspartic acid: | glycine: | valine: | glutamic acid | (DGVE) |
| aspartic acid: | glycine: | valine: | glutamine | (DGVQ) |
| aspartic acid: | glycine: | threonine: | glutamic acid | (DGTE) |
| aspartic acid: | glycine: | threonine: | glutamine | (DGTQ) |
| aspartic acid: | glycine: | serine: | glutamic acid | (DGSE) |
| aspartic acid: | glycine: | serine: | glutamine | (DGSQ) |
| asparagine: | glycine: | leucine: | glutamic acid | (NGLE) |
| asparagine: | glycine: | isoleucine: | glutamic acid | (NGIE) |
| asparagine: | glycine: | valine: | glutamic acid | (NGVE) |
| asparagine: | glycine: | threonine: | glutamic acid | (NGTE) |
| asparagine: | glycine: | serine: | glutamic acid | (NGSE) |
| glutamine: | glycine: | leucine: | glutamic acid | (QGLE) |

Preferred embodiments of the inventions are copolymer compositions comprising a random sequence of one of the following sets of amino acid residues:

aspartic acid, alanine, leucine, and glutamic acid (DALE);

aspartic acid, alanine, isoleucine, and glutamic acid (DAIE);

aspartic acid, alanine, valine, and glutamic acid (DAVE);

aspartic acid, alanine, threonine, and glutamic acid (DATE);

aspartic acid, glycine, leucine, and glutamic acid (DGLE);

aspartic acid, glycine, isoleucine, and glutamic acid (DGIE);

aspartic acid, glycine, valine, and glutamic acid (DGVE); or

aspartic acid, glycine, threonine, and glutamic acid (DGTE).

In general, these compositions are synthesized to have a molar output ratio of amino acid components, as they appear above, of about 1:10:3:1, or 1:15:3:1 respectively. Alternatively, the molar output ratio of amino acid components is about 1:25:15:5, respectively. Alternatively, the molar output ratio of amino acid components is about 1:3:1.5:0.2, respectively. Molar output ratios have a variability range of about 10% between the different amino acids.

In another embodiment, any of the copolymers can further comprise an additional amino acid residue, wherein the copolymer has T cell stimulatory activity in a complex with a class II MHC protein, wherein the additional amino acid residue is found in an autoantigenic peptide for diabetes. For example, an additional amino acid to any of the combination above is a lysine residue (K). The K residue is present in sufficient molar output ratio to increase T-cell stimulation by the copolymer complexed with a class II MHC protein. Further, the K residue present in sufficient molar output ratio to increase aqueous solubility of the copolymer.

A certain ratio of amino acids to be incorporated into the random copolymer may be used. Preferred random copolymers of the present invention comprise amino acid residues K, E, A, S, V, and P. More preferably, the molar input ratio of K:E:A:S:V is 0.3:0.7:9:0.5:0.5:0.3.

Further, in certain embodiments, the copolymer can be a semi-random (or semi-regular) polymer having “anchor,” or fixed, residues which occur with regular spacing in the resulting polymer, providing for optimal class II binding. Preferably, the copolymer has a general sequence:

[XXXa₁XXXXXXXXa₂XX]n,

wherein 2≦n≦8, X is any amino acid residue, and Xa₁ and Xa₂ are acidic amino acid residues selected from glutamic acid and aspartic acid. Alternatively, one of Xa₁ and Xa₂ may be a valine.

Preferably, the copolymer can be synthesized to have one of the general sequences:

1. [XXEXXXXXXXEXX]ₙ

2. [XXEXXXXXXXDXX]ₙ

3. [XXDXXXXXXXDXX]ₙ

4. [XXDXXXXXXXEXX]ₙ

5. [XXEXXVXXXXDXX]ₙ

6. [XXDXXVXXXXDXX]ₙ

7. [XXDXXVXXXXEXX]ₙ

8. [XXEXXVXXXXEXX]ₙ

wherein X is A, S, V, K, or P.

In a preferred embodiment, the molar input ratio of A:S:V:K:P is 5:1:1:1:0.5, and 2≦n≦8. In a preferred embodiment, n=4.

The peptides may have a length of 9 to 25 amino acid residues. Preferably, the peptide is 13 amino acid-residues long. A peptide of a defined sequence length of 9 to 25 amino acids may contain from 2 to 20 fixed residues. An individual fixed residue of a peptide described in this invention may bind to the peptide binding grove of a class II MCH molecule at any of the positions P1, P4, P7, or P9. Preferably, such peptide contains 2 or 3 fixed residues. In one embodiment, a peptide of a defined sequence length of 13 amino acids will contain 2 fixed residues, either E or D or any combination thereof. Preferably a peptide of a defined sequence length of 13 amino acids will contain 3 fixed residues. The peptides may be multimers of a defined sequence, wherein the number of the repeating units preferably ranges from 2 to 8. More preferably, the number of the repeating units is 3 to 6. Most preferably, the number of repeating units is 4. In a preferred embodiment, a multimer of the instant invention comprises a peptide of a defined sequence length of 13 amino acids containing 2 fixed residues, either E or D or any combination thereof.

In preferred embodiments, the copolymers compositions of the present invention bind to one or more DQ isotypes with an average K_{d} of 1 μM or less, and more preferably an average K_{d} less than 100 nM, 10 nM or even 1 nM. Another way to identify preferred copolymers is based on competitive binding assays, such as described in Sidney et al. (2002) J. Immunol. 169: 5098, which is expressed as an IC₅₀ value, or a value of the competitor at which 50% of the binding is inhibited. Preferred copolymers of the present invention have IC₅₀'s less than 1 μM, more preferably less than 500 nM, and even more less than 100 nM.

The copolymer provided herein is at least about 30 residues in length, at least about 40 residues in length, or the copolymer is at least about 50 residues in length. Further, the copolymer is no greater than about 90 residues in length, no greater than about 80 residues in length, or no greater than about 70 residues in length. Preferably, the random copolymers are about 10 to 100 amino acid residues long, more preferably 20 to 80 amino acid residues long, even more preferably 40 to 60 amino acid residues long, and most preferably about 50 amino acid residues long. When synthesized, a typical preparation of random copolymers is a mixture of peptides of various lengths, the majority of which are of the desired length but containing shorter or longer peptides inevitably created by the currently available synthetic processes. Preferably, the peptides are synthesized by solid phase chemistry.

In certain preferred embodiments, the subject copolymers are formulated for use as a medicament so as to have a polydispersity less than 25,000, and more preferably less than 10000, 5000 or even 1000.

The compounds of the invention which reduce HLA-DQ mediated autoimmune responses have therapeutic value in the prevention or treatment of various class II MHC-related diseases or disorders such as Insulin-dependent diabetes mellitus (IDDM), celiac disease, dermatitis herpetiformis and autoimmune thyroid disease (AITD). The compounds of the invention may be administered to a patient for treatment of an immune disorder, for example, involving undesirable or inappropriate immune activity, or may be used to prepare a therapeutic medicament. In particular, an effective dose of a compound of the invention may be therapeutically applied to ameliorate or to prevent insulin-dependent diabetes, celiac disease, and other diseases. An effective dose of a compound of the invention for the treatment of a disorder involving undesirable or inappropriate MHC activity, such as an autoimmune disorder, can be determined by standard means known in the art taking into account routine safety studies, toxicity studies, dose concentration studies and method of delivery, e.g., bolus, continuous or repeated.

### Preparation of Compounds

The compounds of the present invention are random or semi-random copolymers of amino acids residues described above or analogs thereof (such as to form peptidomimetics), which can be synthesized using readily available technology and materials. To illustrate, a copolymer of the invention can be synthesized using Fmoc or t-boc initiating amino acid analogs, or the like, which are immobilized on a resin in an automated peptide synthesis apparatus for further polymerization (solid state synthesis). The amino acids are polymerized in molar ratios that can be adjusted to provide a copolymer with optimal binding characteristics.

Examples of such resin supports for peptide synthesis include a Merrifield resin, chloromethylated polystyrene with 1% DVB cross-links; an Fmoc amino acid Wang resin, 4-benzyloxybenzyl alcohol, the resins being pre-loaded with an amino acid (for example, Fmoc-D-trp(boc)-Wang resin). Resins are available in different mesh sizes, for example 100-200 mesh, and high loading or low loading densities of fractionalization of the initiating amino acid.

Synthesis procedures can include providing a solution which is a mixture of the chosen amino acids in an activated form, for example, activated as an N-carboxy anhydride, in the appropriate molar ratios of each of the appropriately derivatized amino acid precursors (derivatized to protect certain functional groups, such as the e amino group of L-lysine, for example the precursor ∈,N-trifluoroacetyl-L-lysine). Alternatively, the synthesis procedure can involve online mixing during the synthetic procedure of derivatized precursors of the selected amino acids in the preferred molar ratios. The molar output ratio of the amino acids differs from the molar input ratio, that is, the molar ratio of amino acids used in the synthesis mixture differs from the molar ratio of amino acids in the synthesized random copolymer. The molar output ratio is determined by a routine amino acid composition analysis after hydrolysis of the copolymer composition. Molar output ratios have a variability range of about 10% between the different amino acids.

A solution of the different derivatized amino acids to be polymerized into the composition of the invention, preferably protected as conventional in peptide synthesis, is added to sample of beads e.g., Fmoc. Reagents for synthesis, for deblocking, and for cleavage of the complete copolymer molecules for removal from the resin are available from manufacturers of the apparatus (Applied Biosystems Peptide Synthesizer, Foster City, Calif., or Advanced ChemTech, Louisville, Ky.); see e.g., Bodansky, Principles of Peptide Synthesis, 2nd Ed., Springer-Verlag, 1991, the contents of which are herein incorporated by reference. Additional amino acids or analogs or derivatives of amino acids, can be added to the at least three amino acids selected to comprise the copolymers, to substitute for a small proportion of those amino acids, to provide, for example, a copolymer having increased protease resistance and therefore having enhanced pharmacological properties such as longer in vivo lifetime. Examples of analogs are homotyrosine, or other substituted tyrosine derivatives, and aminobutyric acid, each available as an Fmoc derivative from Advanced ChemTech.

Copolymer synthesis services also can be obtained commercially, for example, at Chiron Technologies, Clayton, Australia, the Harvard Medical School Biopolymer Laboratory, Boston, Mass., and at Advanced ChemTech, Inc., Louisville, Ky.

In certain embodiments, the compounds of the present invention include such linear copolymers that are further modified by substituting or appending different chemical moieties. In one embodiment, such modification is at a residue location and in an amount sufficient to inhibit proteolytic degradation of the copolymer in a subject. For example, the amino acid modification may be the presence in the sequence of at least one proline residue; the residue is present in at least one of carboxy- and amino termini; further, the proline can be present within four residues of at least one of the carboxy- and amino-termini. Further, the amino acid modification may be the presence of a D-amino acid.

In certain embodiments, the subject copolymer is a peptidomimetic. Peptidomimetics are compounds based on, or derived from, peptides and proteins. The copolymer peptidomimetics of the present invention typically can be obtained by structural modification of one or more native amino acid residues, e.g., using unnatural amino acids, conformational restraints, isosteric replacement, and the like. The subject peptidomimetics constitute the continuum of structural space between peptides and non-peptide synthetic structures.

Such peptidomimetics can have such attributes as being non-hydrolyzable (e.g., increased stability against proteases or other physiological conditions which degrade the corresponding peptide copolymers), increased specificity and/or potency. For illustrative purposes, peptide analogs of the present invention can be generated using, for example, benzodiazepines (e.g., see Freidinger et al. in “Peptides: Chemistry and Biology,” G. R. Marshall ed., ESCOM Publisher: Leiden, Netherlands, 1988), substituted gamma lactam rings (Garvey et al. in “Peptides: Chemistry and Biology,” G. R. Marshall ed., ESCOM Publisher: Leiden, Netherlands, 1988, p 123), C-7 mimics (Huffman et a. in “Peptides: Chemistry and Biology,” G. R. Marshall ed., ESCOM Publisher: Leiden, Netherlands, 1988, p. 105), keto-methylene pseudopeptides (Ewenson et al. (1986) J. Med. Chem. 29: 295; and Ewenson et al. in “Peptides: Structure and Function (Proceedings of the 9th American Peptide Symposium),” Pierce Chemical Co. Rockland, Ill., 1985), β-turn dipeptide cores (Nagai et al. (1985) Tetrahedron Lett. 26: 647; and Sato et al. (1986) J. Chem. Soc. Perkin Trans. 1: 1231), β-aminoalcohols (Gordon et al. (1985) Biochem. Biophys. Res. Commun. 126: 419; and Dann et al. (1986) Biochem. Biophys. Res. Commun. 134: 71), diaminoketones (Natarajan et al (1984) Biochem. Biophys. Res. Commun. 124: 141), and methyleneamino-modified (Roark et al. in “Peptides: Chemistry and Biology,” G. R. Marshall ed., ESCOM Publisher: Leiden, Netherlands, 1988, p 134). Also, see generally, Session III: Analytic and synthetic methods, in “Peptides: Chemistry and Biology,” G. R. Marshall ed., ESCOM Publisher: Leiden, Netherlands, 1988).

In addition to a variety of side chain replacements which can be carried out to generate the subject copolymer peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

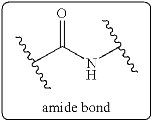

Examples of Surrogates

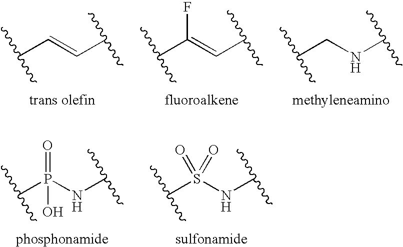

Additionally, peptidominietics based on more substantial modifications of the backbone of the copolymer can be used. Peptidomimetics which fall in this category include (i) retro-inverso analogs, and (ii) N-alkyl glycine analogs (so-called peptoids).

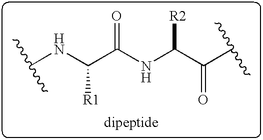

Examples of Analogs

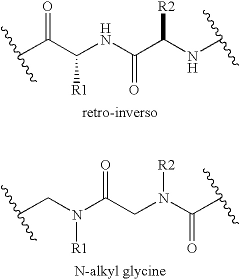

Furthermore, the methods of combinatorial chemistry are being brought to bear on the development of peptidomimetic copolymers. For example, one embodiment of a so-called “peptide morphing” strategy focuses on the random generation of a library of peptide analogs that comprise a wide range of peptide bond substitutes.

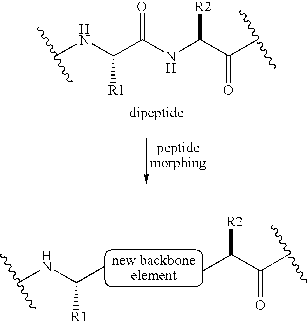

In an exemplary embodiment, the peptidomimetic can be derived as a retro-inverso analog. Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching The final product, or intermediates thereof, can be purified by HPLC.

In another illustrative embodiment, the peptidomimetic can be derived as a retro-enantio copolymer. Retro-enantio analogs such as this can be synthesized commercially available D-amino acids (or analogs thereof) and standard solid- or solution-phase peptide-synthesis techniques.

In still another illustrative embodiment, trans-olefin derivatives can be made. A trans-olefin analog of a copolymer can be synthesized according to the method of Shue et al. (1987) Tetrahedron Lett. 28: 3225 and also according to other methods known in the art. It will be appreciated that variations in the cited procedure, or other procedures available, may be necessary according to the nature of the reagent used.

It is further possible to couple the pseudodipeptides synthesized by the above method to other pseudodipeptides, to make copolymers with several olefinic functionalities in place of amide functionalities. For example, pseudodipeptides corresponding to certain di-peptide sequences could be made and then coupled together by standard techniques to yield an analog of the copolymer peptide which has alternating olefinic bonds between residues.

Still another class of peptidomimetic derivatives includes phosphonate derivatives. The synthesis of such phosphonate derivatives can be adapted from known synthesis schemes. See, for example, Loots et al. in “Peptides: Chemistry and Biology,” (Escom Science Publishers, Leiden, 1988, p. 118); Petrillo et al. in “Peptides: Structure and Function (Proceedings of the 9th American Peptide Symposium),” Pierce Chemical Co. Rockland, Ill., 1985).

In other embodiments, the modification may be introduction of carbohydrate or lipid moieties. Such modifications also change the solubility of the copolymers into various medium so that they may advantageously be prepared into a suitable pharmaceutical composition. Modifying lipid groups include farnesyl group or myristoyl group. Modifying carbohydrate groups include single sugars or oligosaccharides of any naturally occurring and synthetic sugar and sugar alcohols, for example glucose, galactose, rhamnose, mannose, arabinose, and other sugars, and their respective alcohols.

### Method of Treatment

One aspect of the present invention provides methods to treat a subject having an autoimmune disease by administering one or more copolymers of the present invention to the subject in a therapeutically effective amount. Other aspects of the present invention provides methods to treat a subject having an unwanted immune response, allergy, or any disease treatable by administering a copolymer of the invention described herein. The method of treatment provided by the present invention is particularly suitable for treatment of Type I, or insulin-dependent, diabetes mellitus, celiac disease, or any other autoimmune disease mediated through HLA-DQ molecules.

In general, an embodiment of the invention is to administer a suitable daily dose of a therapeutic copolymer composition that will be the lowest effective dose to produce a therapeutic effect, for example, mitigating symptom. The therapeutic copolymers are preferably administered at a dose per subject per day of at least about 2 mg, at least about 5 mg, at least about 10 mg, or at least about 20 mg as appropriate minimal starting dosages. In one embodiment of the methods described herein, a dose of about 0.01 to about 500 mg/kg can be administered. In general, the effective dosage of the compound of the present invention is about 50 to about 400 micrograms of the compound per kilogram of the subject per day. However, it is understood by one skilled in the art that the dose of the composition of the invention will vary depending on the subject and upon the particular route of administration used. It is routine in the art to adjust the dosage to suit the individual subjects. For example, a single bolus can be administered, several divided doses can be administered over time, or the dose can be proportionally reduced or increased as indicated by the exigencies of the disease situation. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point.

An improvement in the symptoms as a result of such administration is noted by a decrease in frequency of recurrences of episodes of diabetes, by decrease in severity of symptoms, and by elimination of recurrent episodes for a period of time after the start of administration. A therapeutically effective dosage preferably reduces symptoms and frequency of recurrences by at least about 20%, for example, by at least about 40%, by at least about 60%, and by at least about 80%, or by about 100% elimination of one or more symptoms, or elimination of recurrences of the autoimmune disease, relative to untreated subjects.

The compound may be delivered hourly, daily, weekly, monthly, yearly (e.g., in a time release form) or as a one-time delivery. The delivery may be continuous delivery for a period of time, e.g., intravenous delivery. In one embodiment of the methods described herein, the agent is administered at least once per day. In one embodiment, the agent is administered daily. In one embodiment, the agent is administered every other day. In one embodiment, the agent is administered every 6 to 8 days. In one embodiment, the agent is administered weekly. The period of time of treatment can be at least about one month, at least about six months, or at least about one year.

In one embodiment of the methods described herein, the route of administration can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art as one skilled in the art may easily perceive. Other embodiments of the compositions of the invention incorporate particulate forms protective coatings, protease inhibitors or permeation enhancers for various routes of administration, including parenteral, pulmonary, nasal and oral.

An embodiment of the method of present invention is to administer the copolymers of the present invention in a sustained release form. Such method comprises applying a sustained-release transdermal patch or implanting a sustained-release capsule or a coated implantable medical device so that a therapeutically effective dose of the copolymer of the present invention is continuously delivered to a subject of such a method. The compounds and/or agents of the subject invention may be delivered via a capsule which allows sustained-release of the agent or the peptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines), or microencapsulated delivery systems. In certain embodiments, a source of a copolymer is stereotactically provided within or proximate to the area of autoimmune attack, for example, near the pancreas for the treatment of IDDM.

In another related embodiment, the methods further comprise administering at least one additional therapeutic agent. Such an agent can be another copolymer such as Copaxone® that binds to a different HLA molecule, which may be an HLA-DQ molecule or an HLA-DR molecule; an antibody or a fragment of an antibody which binds to an unwanted inflammatory molecule or cytokine such as interleukin-6, interleukin-8, granulocyte macrophage colony stimulating factor, and tumor necrosis factor-α; an enzyme inhibitor such as a protease inhibitor such as α₁-antitrypsin, aprotinin, inhibitor of a kallikrein; a cyclooxygenase inhibitor; an antibiotic such as amoxicillin, rifampicin, erythromycin; an antiviral agent such as acyclovir; a steroidal anti-inflammatory such as a glucocorticoid; sex steroid such as progesterone; a non-steroidal anti-inflammatory such as aspirin, ibuprofen, or acetaminophen; an anti-cancer agent such as methotrexate or adriamycin; a cytokine blocking agent; an adhesion molecule blocking agent; an immunosuppressant such as FK506 or cyclosporine; or a non-inflammatory cytokine such as interleukin-4 or interleukin-10. Other cytokines and growth factors may be interferon-β, tumor necrosis factors, antiangiogenic factors, erythropoietins, thrombopoietins, interleukins, maturation factors, chemotactic protein, and their variants and derivatives that retain similar physiological activities.

Another embodiment of the methods of invention further comprises administration of anti-obesity drugs. Anti-obesity drugs include P-3 agonists, CB-1 antagonists, appetite suppressants, such as, for example, sibutramine (Meridia), and lipase inhibitors, such as, for example, orlistat (Xenical).

In an embodiment of the methods of the invention, a copolymer of the invention is administered in combination with drugs commonly used to treat lipid disorders in diabetic patients. Such drugs include, but are not limited to, HMG-CoA reductase inhibitors, nicotinic acid, bile acid sequestrants, and fibric acid derivatives.

In yet another embodiment of the methods of the invention, a copolymer of the invention is administered in combination with anti-hypertensive drugs such as β-blockers, cathepsin S inhibitors and ACE inhibitors.

A copolymer of the present invention may be administered with one more of any of the foregoing additional therapeutic agents.

The additional agent or agents may be administered as an added part of the pharmaceutical composition as described below or may be administered as a separate composition, concomitantly or within a time period when the physiological effect of the additional agent overlaps with the physiological effect of the copolymer of the present invention. More specifically, an additional agent may be administered concomitantly or one week, several days, 24 hours, 8 hours, or immediately before the administration of the copolymer. Alternatively, an additional agent may be administered one week, several days, 24 hours, 8 hours, or immediately after the administration of the copolymer.

Another embodiment of the present invention is a method for prophylactically treating a subject at risk of developing an autoimmune disease by administering a copolymer of the present invention, so that the onset of the disease is delayed or prevented. A subject at risk is identified by, for example, determining the genetic susceptibility to an autoimmune disease by testing for alleles of HLA that are associated with such autoimmune disease, and/or based on familial history, or other genetic markers that correlate with such autoimmune disease. Such prophylactic treatment may additionally comprise a second copolymer that binds to a second HLA molecule associated with the autoimmune disease to be treated. The second HLA molecule may be a HLA-DQ or HLA-DR molecule. Preferably, the autoimmune disease to be prophylactically treated is IDDM or celiac disease.

Prophylactic treatments using a copolymer composition of the present invention are also suitable for preventing unwanted immune responses, such as host-graft disease or graft-host disease or graft rejection after organ transplantation. A copolymer of the invention may be administered to a subject prior to, during, and after transplantation, either alone or with traditional immunosuppressant drugs. Such administration may take place one week, several days, 24 hours, 8 hours or immediately before transplantation, and may continue to be administered to a patient after transplantation in a treatment regimen for another 60-100 days, but at least 60 days, after the transplantation day. Prophylactic treatments using a copolymer composition of the invention is also suitable for preventing allergies, or any disease treatable by administration of a copolymer of the present invention.

### Therapeutic Compositions

Another aspect of the present invention provides pharmaceutical compositions comprising a pharmaceutically effective amount of a copolymer composition of the present invention and an acceptable carrier and/or excipients. A pharmaceutically acceptable carrier includes any solvents, dispersion media, or coatings that are physiologically compatible. Preferably, the carrier is suitable for intravenous, intramuscular, oral, intraperitoneal, transdermal, topical, or subcutaneous administration. One exemplary pharmaceutically acceptable carrier is physiological saline. Other pharmaceutically acceptable carriers and their formulations are well-known and generally described in, for example, Remington's Pharmaceutical Science (18^{th} Ed., ed. Gennaro, Mack Publishing Co., Easton, Pa., 1990). Various pharmaceutically acceptable excipients are well-known in the art and can be found in, for example, Handbook of Pharmaceutical Excipients (4^{th} ed., Ed. Rowe et al. Pharmaceutical Press, Washington, D.C.). The composition can be formulated as a solution, microemulsion, liposome, capsule, tablet, or other forms suitable for various routes of administration described above in for the methods of treatment. The active component which comprises the copolymer may be coated in a material to protect it from inactivation by the environment prior to reaching the target site of action.

In other embodiments of the present invention, the pharmaceutical compositions are sustained release formulations. Copolymers of the present invention may be admixed with biologically compatible polymers or matrices which control the release rate of the copolymers into the immediate environment. Controlled or sustained release compositions include formulation in lipophilic depots (e.g., fatty acids, waxes, oils), implants, transdermal patches, and microencapsulated delivery systems. Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. Acceptable carriers include carboxymethyl cellulose (CMC) and modified CMC. See, e.g., Sustained and Controlled Release Drug Delivery Systems, J. R. Robinson, Ed., Marcel Dekker, Inc., NY, 1978.

The pharmaceutical composition may also include additional therapeutically active ingredients. Such additional ingredient can be another copolymer such as Copaxone® that binds to a different HLA molecule, an antibody or a fragment of an antibody which binds to an unwanted inflammatory molecule or cytokine such as interleukin-6, interleukin-8, granulocyte macrophage colony stimulating factor, and tumor necrosis factor-α; an enzyme inhibitor such as a protease inhibitor such as α₁-antitrypsin, aprotinin, inhibitor of a kallikrein; a cyclooxygenase inhibitor; an antibiotic such as amoxicillin, rifampicin, erythromycin; an antiviral agent such as acyclovir; a steroidal anti-inflammatory such as a glucocorticoid; sex steroid such as progesterone; a non-steroidal anti-inflammatory such as aspirin, ibuprofen, or acetaminophen; an anti-cancer agent such as methotrexate or adriamycin; a cytokine blocking agent; an adhesion molecule blocking agent; an immunosuppressant such as FK506 or cyclosporine; or a non-inflammatory cytokine such as interleukin-4 or interleukin-10. Other cytokines and growth factors such as interferon-β, tumor necrosis factors, antiangiogenic factors, erythropoietins, thrombopoietins, interleukins, maturation factors, chemotactic protein, and their variants and derivatives that retain similar physiological activities may also be used as an additional ingredient of the composition of the invention.

An embodiment of the therapeutic composition of the invention may comprise a copolymer in combination with one or more anti-obesity drugs, such as P-3 agonists, CB-1 antagonists, appetite suppressants, such as, for example, sibutramine (Meridia), and lipase inhibitors, such as, for example, orlistat (Xenical).

One or more drugs commonly used to treat lipid disorders in diabetic patients may be the additional therapeutically active ingredients of the composition of the invention. Such drugs include, but are not limited to, HMG-CoA reductase inhibitors, nicotinic acid, bile acid sequestrants, and fibric acid derivatives.

Anti-hypertensive drugs, such as, for example, β-blockers, cathepsin S inhibitors and ACE inhibitors, may be the additional therapeutically active ingredients of the composition of the invention. Examples of β-blockers are: acebutolol, bisoprolol, esmolol, propanolol, atenolol, labetalol, carvedilol, and metoprolol. Examples of ACE inhibitors are: captopril, enalapril, lisinopril, benazepril, fosinopril, ramipril, quinapril, perindopril, trandolapril, and moexipril. Examples of cathepsin S specific inhibitors are: furanone derivatives having a structure represented by Formula (I) below:

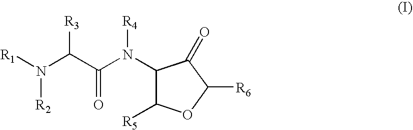

wherein R1═R′, R′C(O), R′C(S), R′SO2, R′OC(O), R′NHC(O), R′═

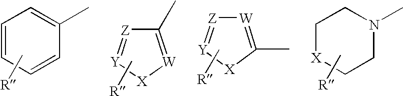

X═O, S, NH, W, Y, Z=CH, N;
R″=single or multiple ring substitution combinations taken from:
H, C1-7-alkyl, C3-6-cycloalkyl, OH, SH, Amine, Halogen;
R2, R4═H, C1-7-alkyl, C3-7-cycloalkyl;
R3=C1-7-alkyl, C3-7-cycloalkyl, Ar—C1-7-alkyl;
R5=C1-7-alkyl, halogen, Ar—C1-7-alkyl, C1-3-alkyl-CONR′″, R^{iv};
R^{iv}═

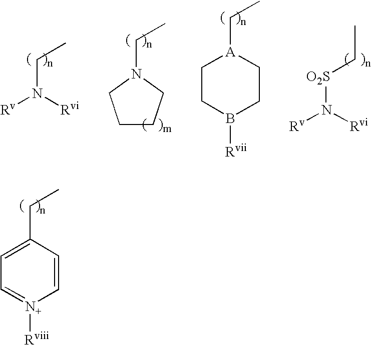

where n=1-3, m=1-3;
R^{v}, R^{vi}═H, C1-7-alkyl;
A=N, CH;
B═N, O, S, CH;
R^{vii}=absent when B═O, S; or R^{vii}═H, C1-7-alkyl when B═N, CH;
R^{viii}═O, C1-7-alkyl;
R6═H, Ar—C1-7-alkyl, C1-3-alkyl-SO2-R^{ix}, C1-3-alkyl-C(O)—NHR^{ix} or CH2XAr;
   where X and Ar are as defined herein; and pharmaceutically acceptable salts thereof. The compounds of formula (I) are disclosed in a published PCT application WO 00/69855, the disclosure of which is incorporated herein in its entirety. Other examples of cathepsin S inhibitors are furanone derivatives having a structure represented by Formula (II) below:

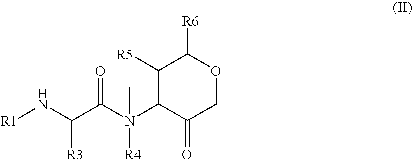

wherein R1 is R′—C(═O)— or R′—S(═O)2-
R′ is

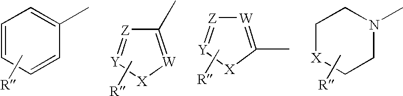

X═O, S, NH,
W,Y,Z=CH, N;
R″=single or multiple ring substitution combinations taken from:
H, C1-7-alkyl, C3-6-cycloalkyl, OH, SH, amine, halogen;
R3═C1-7-alkyl, C2-7-alkenyl, C3-7-cycloalkyl, Ar, Ar—C1-7-alkyl;
R4═H, C1-7-alkyl, C3-7-cycloalkyl; C2-7-alkenyl, Ar, Ar—C1-7-alkyl;
R5═C1-7-alkyl, hydroxyl- or halo-substituted C1-C7-alkylhalogen, Ar—C1-7-alkyl, C0-3-alkyl-CONR3R4 or R^{iv};
R^{iv}═

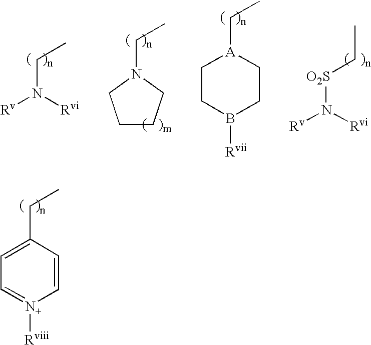

n=1-3, m=1-3;
R^{v}, R^{vi}═H, C1-7-alkyl;
A=N, CH;
B═N, O, S, CH;
R^{vii}=absent when B═O, S; or R^{vii}═H, C1-7-alkyl when B═N, CH;
R^{viii}═O, C1-7-alkyl;
R6═H, C1-7-alkyl, AR—C1-7-alkyl, C1-3-alkyl-SO2—R^{ix}, C1-3-alkyl-C(O)—NHR^{ix} or CH2XAr;
R^{ix} is C1-7-alkyl, Ar—C1-7-alkyl, C3-C6-cycloalkyl and pharmaceutically acceptable salts thereof. The compounds of Formula (II) are disclosed in a published PCT application WO 02/40462, the disclosure of which is incorporated herein in its entirety. Examples of other cathepsin S inhibitors are: 1-[3-[4-(6-Chloro-2,3-dihydro-3-methyl-2-oxo-1H-benzimidazol-1-yl)-1-piperidinyl]propyl]-4,5,6,7-tetrahydro-5-(methylsulfonyl)-3-[4-(trifluoromethyl)phenyl]-1H-pyrazolo[4,3-c]pyridine (JNJ 10329670) (Thurmond et al. (2004) J. Pharmacol. Exp. Ther. 308(1): 268-76, Epub 2003 October 17); CLIK-60 (Katunuma et al. FEBS Lett. 458: 6-10); 4-morpholineurea-Leu-HomoPhe-vinylsulphone (Flannery et al. (2003) Am. J. Pathol. 163(1): 175-82); Paecilopeptin (Shindo et al. (2002) Biosci. Biotechnol. Biochem. 66(11): 2444-8); dipeptide nitriles (Ward et al. (2002) J. Med. Chem. 45(25): 5471-82); and dipeptide alpha-keto-beta-aldehydes (Walker et al. (2000) Biochem. Biophys. Res. Commun. 275(2): 401-5).

The pharmaceutical composition of the present invention is preferably sterile and non-pyrogenic at the time of delivery, and is preferably stable under the conditions of manufacture and storage.

### Method of Identify Therapeutically Active Copolymers

Another aspect of the present invention provides methods for identifying a therapeutic copolymer capable of reducing severity and frequency of episodes of an autoimmune disease.

In certain embodiments, the subject DQ-directed copolymers are modified, or labeled, with a moiety that facilitates the detection of the copolymers. In a preferred embodiment, the copolymers are biotinylated. In another preferred embodiment, the copolymers are modified with FITC. Exemplary copolymers are random copolymers as described above, modified with biotin or FITC. In other embodiments, the copolymers with “anchor” residues which occur with regular spacing in the resulting polymer are modified with biotin or FITC.

In a preferred embodiment, modified copolymers can be synthesized to have one of the general formulae:

9. Biotin-spacer-[XXEXXXXXXEXX]n

10. Biotin-spacer-[XXEXXXXXXXDXX]n

11. Biotin-spacer-[XXDXXXXXXXDXX]n

12. Biotin-spacer-[XXDXXXXXXXEXX]n

13. Biotin-spacer-[XXEXXVXXXXDXX]n

14. Biotin-spacer-[XXDXXVXXXXDXX]n

15. Biotin-spacer-[XXDXXVXXXXEXX]n

16. Biotin-spacer-[XXEXXVXXXXEXX]n

wherein A, S, V, K, or P, the molar input ratio of which are 5:1:1:1:0.5, 2≦n≦8, and the spacer comprises two to 6 amino acid residues, preferably with the amino acid sequence SGSG. In a preferred embodiment, n=4.

These modified copolymers are used in assays and diagnostics, for example in enzyme-linked immunosorbent assay (ELISA). The labeled copolymers can also be used to determine the best sequence or preferred sequence among the copolymers that bind to an HLA molecule. Additionally, the labeled copolymer can be used in screening for other compounds not related to copolymers of the present invention but binds or associate with HLA-DQ molecules.

A copolymer that is therapeutically effective to treat autoimmune disease can be identified by the following method: (1) a copolymer of the present invention is synthesized as described above; (2) determining binding of such copolymer to an HLA-DQ molecule; (3) comparing binding of the copolymer to the HLA-DQ molecule with binding of a known autoantigenic peptide to the HLA-DQ; (4) selecting a copolymer which binds to the HLA-DQ molecule substantially more strongly than the tested known autoantigenic peptide; and (5) determining activation of and anti-inflammatory cytokine production by T helper cells moderated by the HLA-DQ molecule presenting such selected copolymer.

Examples of an autoantigenic peptide are: a peptide comprising amino acid residues 9-23 of human insulin; a peptide comprising amino acid residues 206-220 of human GAD; or a peptide comprising amino acid residues 441-460 of human HSP60. The HLA-DQ molecule that the copolymer is tested against may be any HLA-DQ molecule described herein.

The methods of screening can be used for in vivo assay in non-human animals such as a rodent, such as a rat, mouse, or hamster. The rodent may be a model for human diseases, such as the NOD mice for human diabetes.

### EXAMPLE 1

### Binding of Copolymers to HLA-DQ

The ability of these new copolymers to bind to HLA-DQ molecules are tested by competitive binding assays in which the copolymer competes with a peptide derived from islet autoantigen, examples of which are listed above, for binding to soluble, recombinant HLA-DQ8 molecule. The soluble recombinant HLA-DQ8 encoded by alleles DQA1*03-DQB1*0302 was expressed in Drosophila melanogaster S2 cells under the control of the copper-inducible metallothionein promoter. HLA-DQ8 was engineered to be a soluble protein by replacing the transmembrane and intracellular segments of DQ α and DQ β with leucine zipper dimerization domains from the transcription factors Fos and Jun. See Hausmann et al. (1999) J. Exp. Med. 189: 1723-1734. The expressed recombinant protein was purified from the concentrated supernatants by affinity chromatography using monoclonal antibody 9.3.F10 (HB 180, American Type Culture Collection) and anion-exchange chromatography using Mono Q HR column (Pharmacia Biotech).

Each copolymer was separated and pool sequenced as described in Fridkis-Hareli et al. (1999) J. Immunol. 162: 4697-4704. Generally, fractionation of the polymers was by microbore HPLC using a Zorbax C18 1.0 mm reverse-phase column, eluted with a gradient of 0.055% trifluoroacetic acid in acetonitrile gradient of 0 to 60%. Peak selection, separation by reverse phase, and Edman microsequencing were devised based on Chicz et al. (1993) J. Exp. Med. 178: 27-47.

Copolymer binding assays were performed with biotinylated copolymers. In a binding assay, copolymer candidates and soluble HLA-DQ8 were incubated and the formed complex was captured using monoclonal antibody 9.3.F10, which binds specifically to HLA-DQ8. The captured complex was quantitated by detection with europium-labeled streptavidin. In a competition assay, biotinylated copolymers were preincubated with soluble HLA-DQ8. An unlabeled competitor peptide having an amino acid sequence derived from an islet autoantigen (such as insulin amino acid residues 9-23, GAD amino acid residues 206-220, or HSP60 amino acid residues 441-460) in excess amounts was then added to displace the copolymer. The fraction of copolymer-HLA-DQ8 complexes remaining was calculated from amount remaining bound less non-specific control amounts, measured up to 72 hours to determine the half-life of such complexes.

Copolymers with binding affinity stronger than or comparative to the autoantigenic peptides are selected. Preferred copolymers form complexes with HLA-DQ8 with a half-life of longer than 12 hours. More preferably, copolymers to be selected form complexes with HLA-DQ8 with a half-life of longer than 24 hours, 48 hours, or even more preferably, 72 hours.

The copolymers used in the experiments have the following amino acid composition and, if applicable, anchor amino acids:

RSP-001: [XXEXXXXXXXEXX]₄

RSP-002: [XXEXXXXXXXDXX]₄

RSP-003: [XXEXXVXXXXDXX]₄

Wherein X is a mixture of A, K, S, V, P with a molar input ratio of 5:1:1:1:0.5 at the start of the synthesis.

RSP-008: a random mixture of DAVE

RSP-009: a random mixture of DATE

RSP-010: a random mixture of DALE

Wherein the molar input ratio of the four amino acids is 1:5:3:1 at the start of the synthesis.

CO-14: YFAK with the molar input ratio of the four amino acid residues about 1:1.2:18:6 in the resulting random copolymer composition.

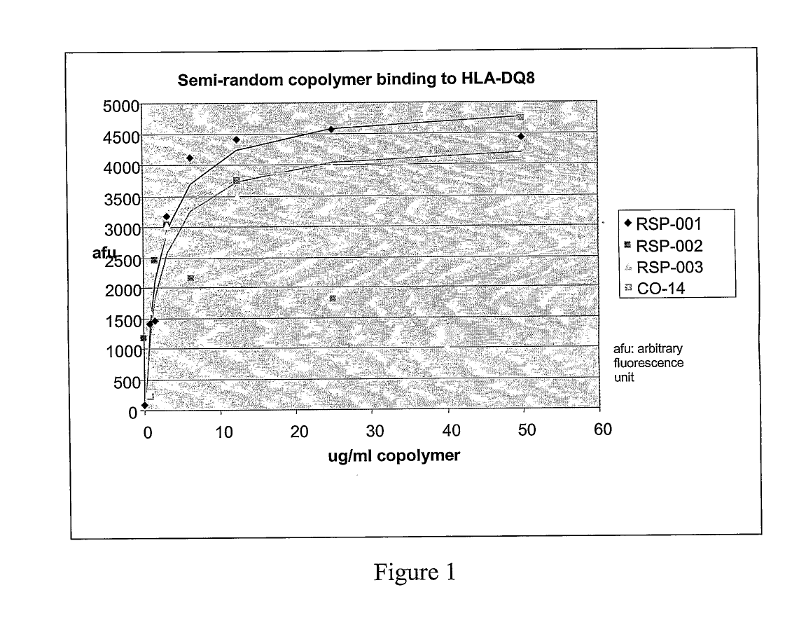

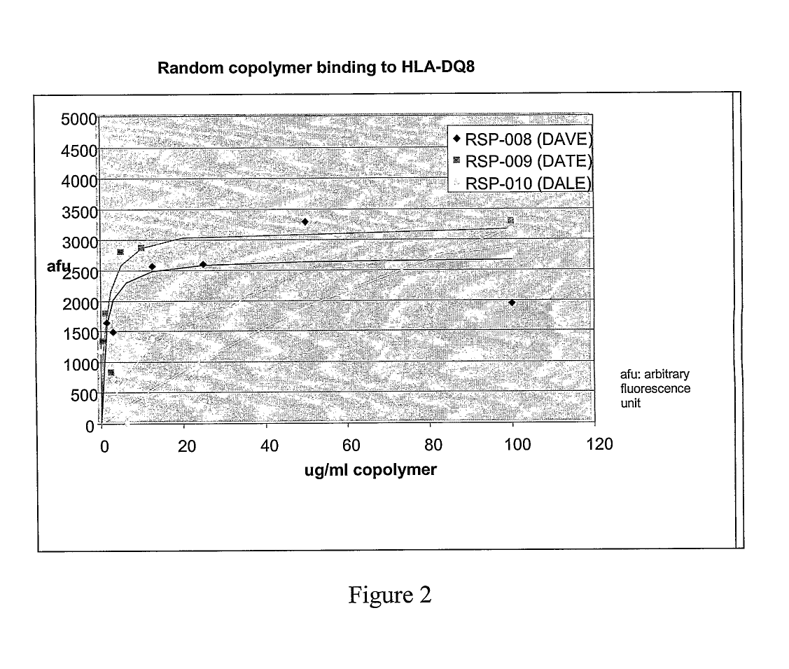

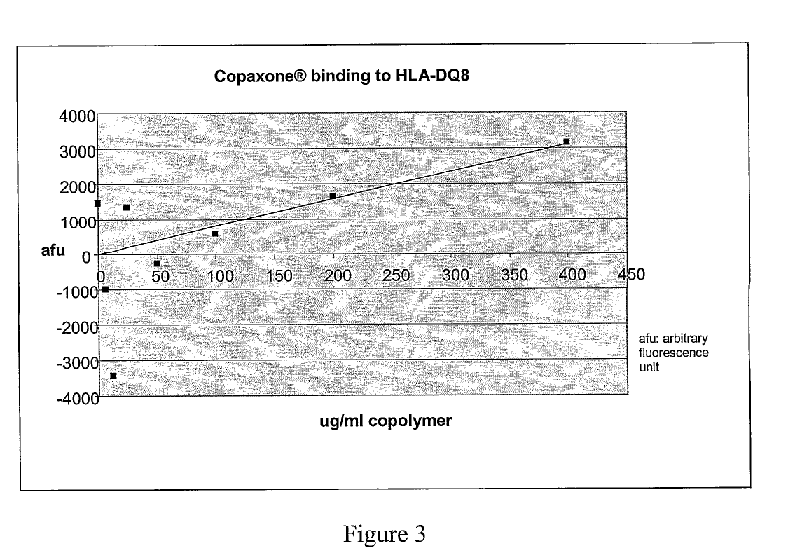

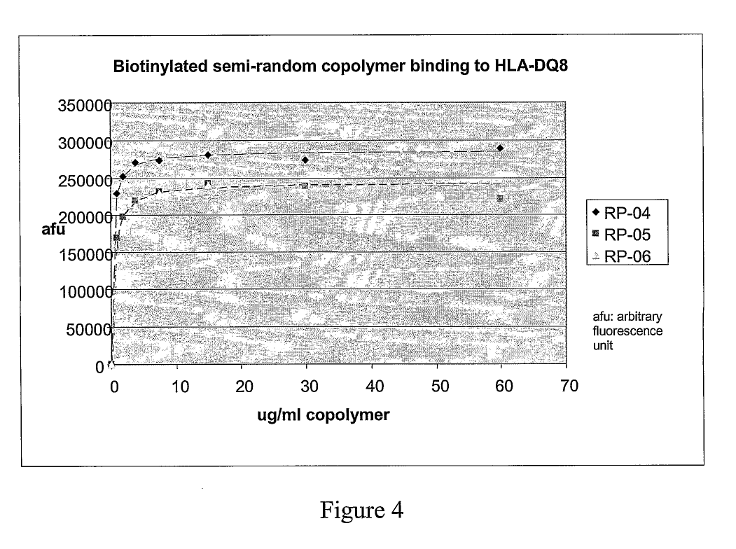

The results of the binding assays are shown in FIGS. 1-6. FIG. 1 shows the results of a competition assay, where non-labeled random copolymers RSP-001, RSP-002 and RSP-003 competed for binding to HLA-DQ8 with RSP-006, which is a biotinylated RSP-003. FIG. 2 shows the results obtained using the competition assay for non-labeled random copolymers RSP-008 (DAVE), RSP-009 (DATE), and RSP-010 (DALE) binding to HLA-DQ8 in competition with RSP-006. FIG. 3 shows the results obtained using the competition assay for CO-14 (YFAK), a copolymer composition originally of interest because of its affinity to HLA-DR molecules. The graphs show results that are corrected for the nonspecific control, and shows the amounts of random copolymers that were displaced. FIG. 4 shows the results of a direct binding assay of biotinylated random copolymers RSP-004 (biotinylated RSP-001), RSP-005 (biotinylated RSP-002), and RSP-006 (biotinylated RSP-006). The results obtained from these experiments consistently show that these random copolymers bind to HLA-DQ8 with similar affinities.

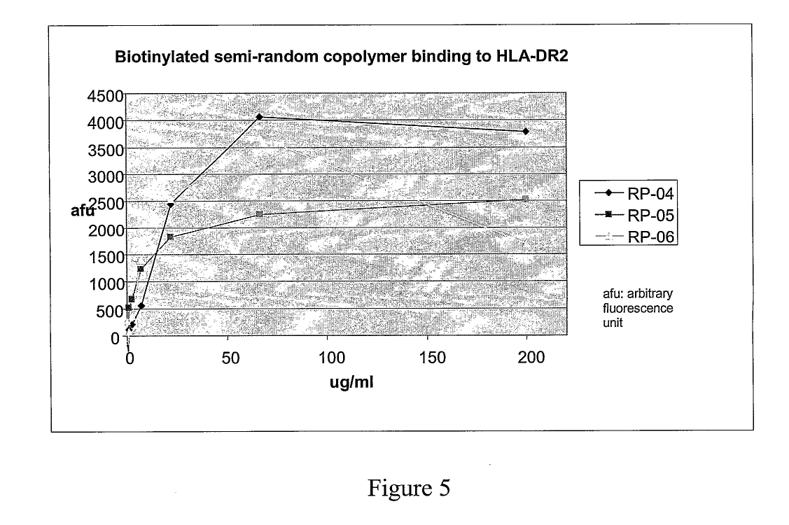

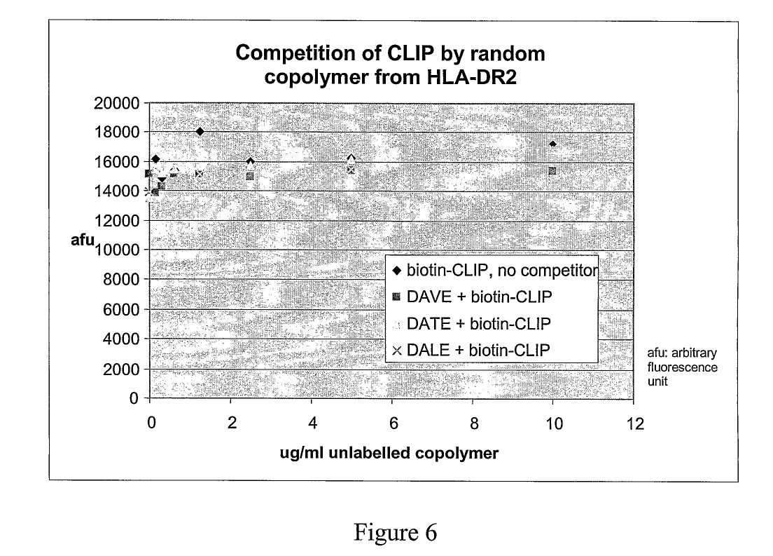

FIGS. 5 and 6 are control experiments showing the binding of these random copolymers to HLA-DR2 protein. FIG. 5 is a direct binding assay of biotinylated random copolymers RSP-004, RSP-005, and RSP-006. The results indicate that these random copolymers are specific to HLA-DQ8 by about a factor of 10. FIG. 6 shows the results of competition assay of RSP-008 (DAVE), RSP-009 (DATE), and RSP-010 (DALE) to HLA-DR2 in competition with CLIP (class II-associated invariant chain peptide; Riberdy et al. (1992) Nature 360(6403):474-7), which binds to various class II proteins with moderate affinity. These results indicate that none of these three random copolymers can compete away biotinylated CLIP, leading to a conclusion that the binding of these random copolymers of the invention is specific to HLA-DQ8.

The dissociation constant K_{d} against HLA-DQ8 and “completeness” were calculated based on the curve obtained from fitting the data observed. The summary of the results are shown in Table 3 below. “Completeness” is defined as the difference between the average fluorescence of the RP-006 peptide in the absence of a competitor and the point at which the binding curve reaches experimental saturation. Completeness is measured as the number of fluorescent units of biotin-labeled peptide that the unlabelled peptide can compete away. It was observed that with each “class” of random copolymers has a consistent “completeness,” suggesting certain subpopulation of the copolymer may bind to the HLA protein by a mode of binding unique to that class of copolymer, and thus cannot be competed out by copolymers of a different class. A “class” of random copolymers comprise copolymer compositions that share structural features such as the same anchor residues or comparable amino acid composition. This is suggested by the observation that RP-006 peptide can more completely displace RSP-001, 002, and 003, which are in the same class of copolymers as RP-006, but cannot displace other copolymers such as RSP-008, 009 or 010 as efficiently.

Additionally, it is calculated that the dissociation constants of the random copolymers tested against HLA-DR2 are about 10-20 μg/ml, and thus about 10 times larger than the values for HLA-DQ8.

TABLE 3
Binding of Random Copolymers to HLA-DQ8

| Copolymer | Kd (μg/ml) | Completeness |
|---|---|---|
| RSP-001 | 2.14 | 4965 |
| RSP-002 | 2.13 | 4401 |
| RSP-003 | 1.33 | 4177 |
| RSP-008 | 1.00 | 2707 |
| (DAVE) | | |
| RSP-009 | 1.22 | 3220 |
| (DATE) | | |
| RSP-010 | 19.18 | 3468 |
| (DALE) | | |
| CO-14 | 2.31 | 2119 |
| Glatiramer | 8148.55 | ND |
| acetate | | |

To test further the ability of the copolymers selected by the binding assays to activate human T cells in a HLA-DQ restricted manner, the copolymers are incubated with human PBMCs from subjects with the HLA-DQ2 encoded by alleles DQA1*0501-DQB1*0201 or HLA-DQ8 encoded by alleles DQA1*03-DQB1*0302 allele. The restriction element(s) for the resulting cell lines can be determined with anti-DR and anti-DQ antibodies.

### EXAMPLE 2

### Peptides Bound to Human Class II MHC HLA-DQ8

Copolymers, peptides and antibodies. Peptides were synthesized using solid phase techniques (Barany, G., and R. Merrifield. 1979. Academic Press, New York, N.Y.) on an Applied Biosystems Peptide Synthesizer and purified by reversed-phase HPLC(RP-HPLC).

A “humanized” mouse model of diabetes in which the mice lack endogenous class II genes but transgenetically express human HLA-DQ8 and DR3 proteins is used in the studies herein. GAD65 is injected into these transgenic mice which are thus immunized with GAD65. HLA-DR3 and DQ8 and their bound peptide fragments from GAD65 are purified from mouse spleen and lymph nodes after the appearance of GAD65 antibody. The obtained peptide pool is fractionated and the T-cells generated from the immunized transgenic mice are tested for their response to these peptides. Whether GAD65 peptides are presented by both DR and DQ proteins in these transgenic animals is determined, and the sequences of the peptides associated with each type of protein is compared, to determine whether they are the same or overlapping peptides. Moreover, whether the presence of one class II MHC protein influences the peptide repertoire present associated with the other protein is determined from comparison of single and double transgenic animals.

Purification of these MHC proteins was achieved using the BioCAD instrument which permits a microscale and rapid purification. The peptide pool was fractioned by HPLC and the peptide peaks of interest were identified by IL-2 production or proliferation assays using the T cell hybridomas generated from the spleens and lymph nodes of the immunized transgenic mice, followed by identification of the peptide in peaks. Finally, the peptides identified are synthesized and mice are immunized with them to determine whether they are immunogenic.

Protein expression and purification. Soluble HLA-DQ molecules were expressed in Drosophila S2 cells and purified as described (Kalandadze et al. 1996. J. Biol. Chem. 271:20156-20162). Cells were grown at 26° C. in roller bottles in ExCell 401 medium (JRH Biosciences, Lenexa, Kans.) supplemented with 0-5% fetal bovine serum (Sigma Chemicals, St. Louis, Mo.). Cells were harvested 4-5 days after induction by 1 mM CuSO₄. Supernatant from harvested cells was sequentially passed through Protein A, Protein G and Protein A-LB3.1 columns, followed by elution of the bound HLA-DR with 50 mM 3-[cyclohexylamino]-1-propanesulfonic acid (CAPS), pH 11.5, and neutralized with 200 mM phosphate (pH 6.0). Proteins were concentrated on a Centriprep 10 membrane (Amicon, Beverly, Mass.).

HPLC separation and microsequencing of bound copolymers. Each of the bound copolymers is separated from unbound material and pool sequenced as previously described (Fridkis-Hareli, M. et al. (1999) J. Immunol. 162:4697-4704). Briefly, the fractionation is by microbore HPLC using a Zorbax C18 1.0 mm reverse-phase column on a Hewlett-Packard 1090 HPLC with 1040 diode array detector. Copolymers are eluted at a flow rate of 54 μl/min with a gradient of 0.055% trifluoroacetic acid (TFA) in acetonitrile (0% at 0 to 10 min, 33% at 73 min and 60% at 105 min). Strategies for peak selection, reverse phase separation and Edman microsequencing have been previously described (Chicz, R. M. et al. (1993) J. Exp Med. 178: 27-47; Godkin et al. (1997) Int. Immunol. 9:905-11). Pooled fractions are submitted to automated Edman degradation on a Hewlett-Packard G1005A (Palo Alto, Calif.) protein sequencer using the manufacturer's Routine 3.5.

Assays for peptide binding to class II MHC proteins. The solutions used in this assay are the following: binding buffer is 20 mM 2-[N-morpholino]ethanesulfonic acid (MES), 140 mM NaCl, 0.05% NaN₃, pH 5.0, unless otherwise specified; PBS is 150 mM sodium chloride, 7.5 mM Na₂HPO₄, 2.5 mM NaH₂PO₄, pH 7.2; TBS is 137 mM sodium chloride, 25 mM Tris pH 8.0, 2.7 mM potassium chloride; TTBS is TBS plus 0.05% Tween-20.

Prior to adding samples, the immunoassay plates (96-well microtiter, PRO-BIND™, Falcon, Lincoln Park, N.J.) were coated with 1 μg/well affinity-purified LB3.1 monoclonal antibodies in PBS (100 μl total) for 18 hrs at 4° C. The wells were then blocked with TBS/3% bovine serum albumin (BSA) for 1 hr at 37° C. and washed three times with TTBS. Immediately before sample addition, 50 μl of TBS/1% BSA was added to each well.

Inhibition reaction was carried out by co-incubating a biotinylated peptide, final concentration 0.13 μM in 50 μl of the binding buffer, with unlabeled inhibitors (random copolymers or control peptides) and HLA-DQ molecules for 40 hr at 37° C. Bound peptide-biotin was detected using streptavidin-conjugated alkaline phosphatase, as follows. Plates were washed three times with TTBS and incubated with 100 μl of streptavidin-conjugated alkaline phosphatase (1:3000, BioRad, Richmond, Calif.) for 1 hr at 37° C., followed by addition of p-nitrophenyl phosphate in triethanolamine buffer (BioRad). Absorbance at 410 nm was monitored by a microplate reader (model MR4000; Dynatech, Chantilly, Va.).

Techniques were used for sequencing of peptides eluted from HLA-DQ8 purified on a small scale. Purification from HLA-DQ8 protein isolated from 1 g of cells. Priess cells (HLA-DR4, HLA-DQ8 homozygous) are the standard in this investigation. POROS immunoaffinity columns (BioCAD instrument) were used to isolate HLA-DQ8 (as well as HLA-DR4) from 20 grams of cells. Peptides were eluted from 1/20 of this material (equivalent of to 1 g of cells).

A total of 79 peptide sequences were identified in the mixture by LC-MS-MS (Table 3). Many of these represented nested peptide sets. One peptide in particular derived from a Class I MHC protein was present in 10 different sequences differing only at the N- or C-terminus of the peptide. The core in this set was readily identified. This represents the largest number of peptides isolated from HLA-DQ8 protein and identified, and this critical number provides a consensus for analysis of such sites on the protein as the P1 and P9 positions (see Table 4). In the previous two studies of HLA-DQ8 (1, 2), sequences of eight peptides were reported in one and the other described only peptide pool sequencing. This analysis was then used with one gram of Priess cells for isolation (1 gram is the amount of spleen that can be obtained from 15-20 mice.). The HLA-DQ8 protein complex having endogenous peptide was readily isolated on this scale, and peptides were readily identified (Table 4). This procedure yielded the sequences of 112 peptides, many more than were previously observed (Chicz et al (1994) Int. Immunol. 6:1639-49; Godkin et al. (1997) Int. Immunol. 9:905). On the basis of such a large number of sequences, it is possible to develop a consensus for binding by the class II MHC HLA-DQ2 encoded by alleles DQA1*0501-DQB1*0201 or HLA-DQ8 encoded by alleles DQA1*03-DQB1*0302 allele.

TABLE 4

Peptide sequences obtained from Priess cell
preparation (20 grams, 1/20 run on LCMSMS)

Proteoglycan

| | | | |
|---|---|---|---|
| 1 | LPSDSQDLGQHGLEEDFM* | (8 | (SEQ ID NO: 10) |
| | | nested) | |
| 2 | IQDLNRIFPLSEDYS | | (SEQ ID NO: 11) |

Class I histocompatibility antigen

| | | | |
|---|---|---|---|
| 3 | TAADTAAQITQRKW | (3 | (SEQ ID NO: 12) |
| | | nested) | |
| 4 | GPEYWDRETQISKTNTQ | (3 | (SEQ ID NO: 13) |
| | | nested) | |
| 5 | PAGDRTFQKWAAVVVPSGEEQR | (10 | (SEQ ID NO: 14) |
| | | nested) | |
| 6 | DTQFVRFDSDAASPRGEP | (3 | (SEQ ID NO: 15) |
| | | nested) | |

MHC class I antigen

| | | |
|---|---|---|
| 7 | LNEDLRSWTAADTAA | (SEQ ID NO: 16) |

MHC class antigen (A302 alpha chain)

| | | | |
|---|---|---|---|
| 8 | VRFDSDAASQRMEPRAP | (4 | (SEQ ID NO: 17) |
| | | nested) | |

Ig kappa chain variable region

| | | | |
|---|---|---|---|
| 9 | IPDRFSGSGSGTDFTLT | (4 | (SEQ ID NO: 18) |
| | | nested) | |
| 10 | GSGTDFTLTISRLEPEDF | (4 | (SEQ ID NO: 19) |
| | | nested) | |

Glyceraldehyde-3-phosphate dehydrogenase

| | | | |
|---|---|---|---|
| 11 | STFDAGAGIALNDH | (3 | (SEQ ID NO: 20) |
| | | nested) | |

Trans-Golgi network integral membrane protein
TGN51 precursor

| | | |
|---|---|---|
| 12 | GPIDGPSKSGAEE | (SEQ ID NO: 4) |
| 13 | TGPEEGSPPKEEKE | (SEQ ID NO: 21) |
| 14 | TPKDGSNKSGAEEQGPI | (SEQ ID NO: 22) |

HLA-DR alpha heavy chain

| | | | |
|---|---|---|---|
| 15 | KFHYLPFLPSTEDVYD | (3 | (SEQ ID NO: 23) |
| | | nested) | |

Unknown

| | | | |
|---|---|---|---|
| 16 | DPGGSVPSGEAS | (3 | (SEQ ID NO: 24) |
| | | nested) | |

Unnamed protein product

| | | | |
|---|---|---|---|
| 17 | LVKGFYPSDIAVEWESN | (4 | (SEQ ID NO: 25) |
| | | nested) | |

CD44 antigen

| | | | |
|---|---|---|---|
| 18 | DGPITITIVNRDGTR | (2 | (SEQ ID NO: 26) |
| | | nested) | |

CD74 antigen invariant polypeptide

| | | | |
|---|---|---|---|
| 19 | PSSGLGVTKQDLGPVM* | (3 | (SEQ ID NO: 27) |
| | | nested) | |

P60 antigen

| | | |
|---|---|---|
| 20 | DEDGDLVAFSSDEE | (SEQ ID NO: 28) |
| 21 | RPGTAESASGPSEDPSVN | (SEQ ID NO: 29) |

Beta-2-microglobulin precursor

| | | | |
|---|---|---|---|
| 22 | LYYTEFTPTEKDEYAC | (2 | (SEQ ID NO: 30) |
| | | nested) | |

Solute carrier family

| | | | |
|---|---|---|---|
| 23 | GPAGDATVASEKES | (2 | (SEQ ID NO: 31) |
| | | nested) | |

Similar to WW domain binding protein 2

| | | | |
|---|---|---|---|
| 24 | GPDVPSTAAEAKA | (2 | (SEQ ID NO: 32) |
| | | nested) | |

Heat shock cognate 71 kDa Protein

| | | | |
|---|---|---|---|
| 25 | INWLDKNQTAEKEEFEH | (2) | (SEQ ID NO: 33) |
| 26 | TGIPPAPRGVPQ | | (SEQ ID NO: 34) |

MHC class I antigen (peromyscus maniculatus)

| | | | |
|---|---|---|---|
| 27 | FQKWAAVVVPTGEE | (2 | (SEQ ID NO: 35) |
| | [only differs by S -> | nested) | |
| | T difference from | | |
| | above class I] | | |

MHC DQ-alpha 1 protein (rhesus macaque)

| | | | |
|---|---|---|---|
| 28 | FPGDEEFYVDLERKET | (2 | (SEQ ID NO: 36) |
| | | nested) | |

Myobrevin (vesicle associated membrane protein 5)

| | | |
|---|---|---|
| 29 | APRTQDAGIASPGPGN | (SEQ ID NO: 37) |

Fc fragment of IgE, low affinity ii, receptor
(CD23A)

| | | |
|---|---|---|
| 30 | AQKSQSTQISQELEE | (SEQ ID NO: 38) |

TABLE 5

Peptide sequences obtained from Priess cell preparation
(1 gram prep)

Proteoglycan 1

| | | | |
|---|---|---|---|
| 1 | SLDRNLPSDSQDLGQHGLEEDFM*L | (15 nested) | (SEQ ID NO: 39) |

Class I histocompatibility antigen

| | | | |
|---|---|---|---|
| 2 | RPAGDGTFQKWAAVVVPSGEEQR | (10 nested) | (SEQ ID NO: 40) |
| 3 | GPEYWDRETQISKTNT | | (SEQ ID NO: 41) |
| 4 | LNEDLSSWTAADTAA | | (SEQ ID NO: 42) |
| P60 | | | |
| 5 | YRDEDGDLVAFSSDEELT | (6 nested) | (SEQ ID NO: 43) |

Calnexin

| | | | |
|---|---|---|---|
| 6 | IDIEDDLDDVIEEVEDSKP | (2) | (SEQ ID NO: 44) |
| 7 | KPDDWDEDAPAKIPDEE | (5 nested) | (SEQ ID NO: 45) |
| 8 | KPEDWDEDM*DGEWEAPG | | (SEQ ID NO: 46) |

Alzheimer's disease amyloid A4 protein homolog precursor

| | | | |
|---|---|---|---|
| 9 | ADGSEDKVVEVAEEEEVA | (7 nested) | (SEQ ID NO: 47) |

Chain A, rabbit serum transferrin

| | | | |
|---|---|---|---|
| 10 | APEEGYLSVAVVK | | (SEQ ID NO: 48) |
| 11 | DLGDVAFVK | (2) | (SEQ ID NO: 49) |
| 12 | SGDFQLFSSPHGK | | (SEQ ID NO: 50) |
| 13 | SQTVLQNTGGR | | (SEQ ID NO: 51) |
| 14 | EGYYGYTGAFR | | (SEQ ID NO: 52) |

Fc fragment of IgE, low affinity IL receptor for (CD23A)

| | | | |
|---|---|---|---|
| 15 | HHGDQM*AQKSQSTQISQELEELRAEQQ | (5 nested) | (SEQ ID NO: 53) |

Heat shock protein gp 96 precursor

| | | | |
|---|---|---|---|
| 16 | KEESDDEAAVEEEEEEKKP | (4 nested) | (SEQ ID NO: 54) |
| 17 | IDPDAKVEEEPEEEPE | | (SEQ ID NO: 55) |

Class II antigen alpha

| | | | |
|---|---|---|---|
| 18 | FDGDEEFYVDLERKETV | (8 nested) | (SEQ ID NO: 56) |
| 19 | EDIVADHVASYGNLYQSYGPSGQYSHEFD | (3) | (SEQ ID NO: 57) |

Ig kappa chain variable region

| | | | |
|---|---|---|---|
| 20 | SGTDFTLTISRLEPEDF | (4 nested) | (SEQ ID NO: 58) |

Calreticulin precursor

| | | | |
|---|---|---|---|
| 21 | EPEEEDVPGQAKDEL | (2 nested) | (SEQ ID NO: 59) |
| 22 | IPDPDAKKPEDWDEEM*DGEWEPP | (5) | (SEQ ID NO: 60) |

N-acetyglucosaminyltransferase III

| | | | |
|---|---|---|---|
| 23 | RPDDVFIIDDADEIPARDGV | (4 nested) | (SEQ ID NO: 61) |

DKFZ566HO73 protein

| | | | |
|---|---|---|---|
| 24 | GPGDEDQEEETQGQEEGDEGEPRDHPA | (4 nested) | (SEQ ID NO: 62) |

CD20 receptor

| | | | |
|---|---|---|---|
| 25 | EKKEQTIEIKEEVVG | | (SEQ ID NO: 63) |
| 26 | PKNEEDIEIIPIQEEEEE | (3 nested) | (SEQ ID NO: 64) |

Heat shock cognate 71 kDa protein

| | | | |
|---|---|---|---|
| 27 | INWLDKNQTAEKEEFEH | (3 nested) | (SEQ ID NO: 65) |

Unknown (protein for MGC: 12802)

| | | |
|---|---|---|
| 28 | ENPEDEPLGPEDEDSFS | (SEQ ID NO: 66) |
| 29 | EPLGPEDEDSFSNAESYE | (SEQ ID NO: 67) |

CA125 ovarian carcinoma antigen

| | | |
|---|---|---|
| 30 | IHIAEEEAVM*EEEEDEE | (SEQ ID NO: 68) |

Ubiquitin (Drosophila)

| | | |
|---|---|---|
| 31 | EPSDTXENVKAKIQDKEG | (SEQ ID NO: 69) |

DP alpha chain precursor, similar to

| | | |
|---|---|---|
| 32 | EFDEDEM*FYVDLDKKET | (SEQ ID NO: 70) |

Heat shock cognate 70 ii(Xenopus)

| | | |
|---|---|---|
| 33 | VVSWLDKNQTAEKEEFEH | (SEQ ID NO: 71) |

Protein disulfide isomerase related protein (calcium-
binding protein, intestinal-related)

| | | |
|---|---|---|
| 34 | SNRENAIEDEEEEEEE | (SEQ ID NO: 72) |

Similar to Y39E4B 7p

| | | |
|---|---|---|
| 35 | DPGIFPRAEEDED | (SEQ ID NO: 73) |

Class I histocompatibility Aw68 1

| | | |
|---|---|---|
| 36 | LYYTEFTPTEKDEYA | (SEQ ID NO: 74) |

MHC surface glycoprotein

| | | |
|---|---|---|
| 37 | RKFHYLPFLPSTEDVYD | (SEQ ID NO: 75) |

Similar to Ig heavy chain 4 (serum IgG1) (mouse)

| | | |
|---|---|---|
| 38 | PEVQFSWPVDDVEVHTA | (SEQ ID NO: 76) |

### EXAMPLE 3

### Analysis of Peptide Sequences

The eluted peptides were found to be generally acidic, with surprising overrepresentation of both aspartic acid (D) and glutamic acid (E). Alignment of the peptides with E or D near the carboxy terminus of the core, i.e., at P9, is shown in Table 6. A preference for an acidic amino acid at P1 was also evident in the alignment which is more than that observed with mouse protein I-A^{g7} (Suri et al. (2002) J. Immunol. 168(3):1235-43). The distinction between preferences from one species to another may be especially significant for immune recognition.

TABLE 6

Alignment of sequences of selected peptides
obtained from complexes with HLA-DQ8 in the
homozygous Priess cell line

| | | | |
|---|---|---|---|
| | | SEQ | # of |
| | | ID | nested |
| Core Peptide | Source Protein | NO | peptides |

| | | | |
|---|---|---|---|
| LPSDSQDLGQHGLEE | proteoglycan 1 | 1 | 23 |
| WAAVVVPSGEE | class I MHC | 2 | 20 |
| FDAGAGIALNDH | glyceraldehyde-3- | 3 | 3 |
| | phosphate | | |
| | dehydrogenase | | |
| GPIDGPSKSGAEE | trans-Golgi network | 4 | 1 |
| | protein (TGN 51) | | |
| DEDGDLVAFSSDEE | p60 | 5 | 7 |
| INWLDKNQTAEKEEFEH | 71 kDa heat shock | 6 | 5 |
| | protein | | |
| LYYTEFTPTEKDEYA | beta-2-microglobulin | 7 | 2 |
| DWDEDAPAKIPDEE | calnexin | 8 | 5 |
| GSEDKVVEVAEEE | amyloid A4 protein | 9 | 7 |
| | homolog | | |

Putative P1 and P9 residues are in bold type. Possible multiple candidates are shown for most of these peptides.

### EXAMPLE 4

### Analysis of Peptide Bound to Mouse Class II MHC

Experiments are conducted to obtain a similar analysis for mouse class II MHC, using I-Ag7 isolated from mouse spleens. This modification to a published procedure using 15 mouse spleens resulted in the isolation of a small amount of I-Ag7 of high purity (Suri et al, (2002) J. Immunol. 168 (3): 1235-43). Peptides are isolated, sequenced, and analyzed as shown above for human class II MHC proteins, to obtain a consensus for the mouse. The microtechnique for isolation of I-Ag7 and purification of peptides is performed as for HLA-DQ8. The above example is conducted with the HLA-DQ8 transgenic mouse and the HLA-DR3/HLA-DQ8 double transgenic mouse. The BDC2.5 TCR, CDld−/− mouse in which the disease develops much more rapidly (Shi et al. (2001) Proc. Natl. Acad. Sci. USA 98: 6777-6782) is also employed.

### EXAMPLE 5

### Copolymers for the Treatment of Diabetes: Copaxone® Control

To develop a copolymer that could prevent progression of diabetes in the Non-obese diabetic (NOD) mouse, the consensus amino acids observed for P1 and P9 positions herein in Table 6 were used as the basis of choice of amino acid residues, i.e., which amino acids to use to obtain a copolymer having a random sequence.

NOD mice are obtained from Jackson Laboratories, Bar Harbor, Me., and are used as an experimental system (Shi et al. (2001) Proc. Natl. Acad. Sci. USA 98: 6777-6782). These mice begin to develop diabetes at about 13-15 weeks and data are obtained after about 30 weeks to obtain data, such as a frequency of symptoms for comparison of treated and untreated groups of mice. NOD mice were treated herein with Copaxone®, and no difference was found between the untreated animals and animals treated with Copaxone®. In the experiment, 10 μg of Copaxone® was injected into mice three times a week (equivalent to the human dose on a weight ratio basis. A higher dose, 33 μg Copaxone®/mouse three times a week, is also employed. These experiments test the hypothesis that Copaxone® would be ineffective.

New copolymers based on the binding motif in Table 6 are designed, synthesized, and tested. The composition of these copolymers is given in the Summary herein.

### EXAMPLE 6

### T Cell Recall Response against Random Copolymers

NOD mouse is a widely studied murine model for human insulin-dependent diabetes mellitus. In NOD mice, I-A^{g7} is expressed, a class II MHC molecule that shares structural and peptide-binding similarities with HLA-DQ8, and in mouse confers susceptibility to IDDM. Experiments were carried out to determine whether administrations of semi-random copolymers which contain anchor residues, can prime NOD mice so that the mice will generate T cell responses that can be measured by in vitro T cell assays.

NOD/Ltj mice were immunized by subcutaneous injection between the shoulders on Day 1 with 50 or 250 μg of RSP-001, RSP-002, RSP-003, or RSP-010, alone or with complete Freund's adjuvant (CFA). For RSP-001, 002 and 003, on days 3, 5, 8, 10 and 12, the same subcutaneous injection was repeated as booster immunization. For RSP-010, the same injection was repeated on day 8. Control group animals were injected with sodium phosphate buffer. On day 15, mice were sacrificed and spleens were collected. Splenocytes were restimulated in vitro with various concentrations of same copolymer used for immunization. At least 3 replicates were plated for each treatment group for restimulation. On day 2 of culture, tritiated thymidine was added to triplicate wells, and the cells were harvested on day 3 to measure the incorporation of radioactivity, which shows the proliferation of the primed T cells in response to an antigen.

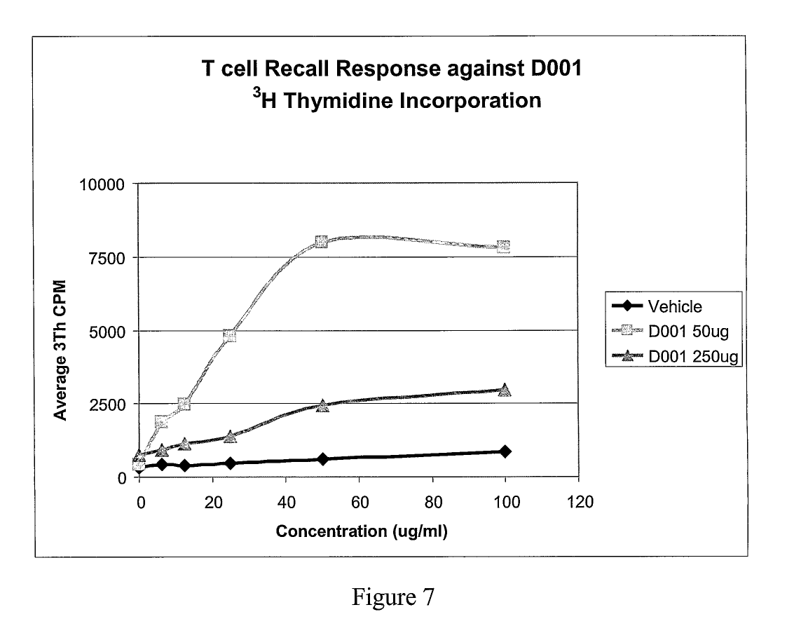

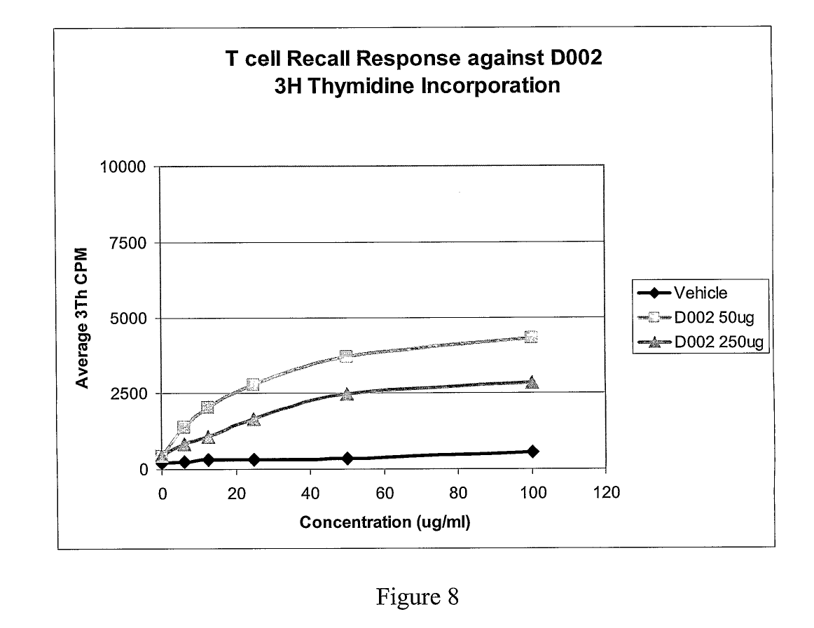

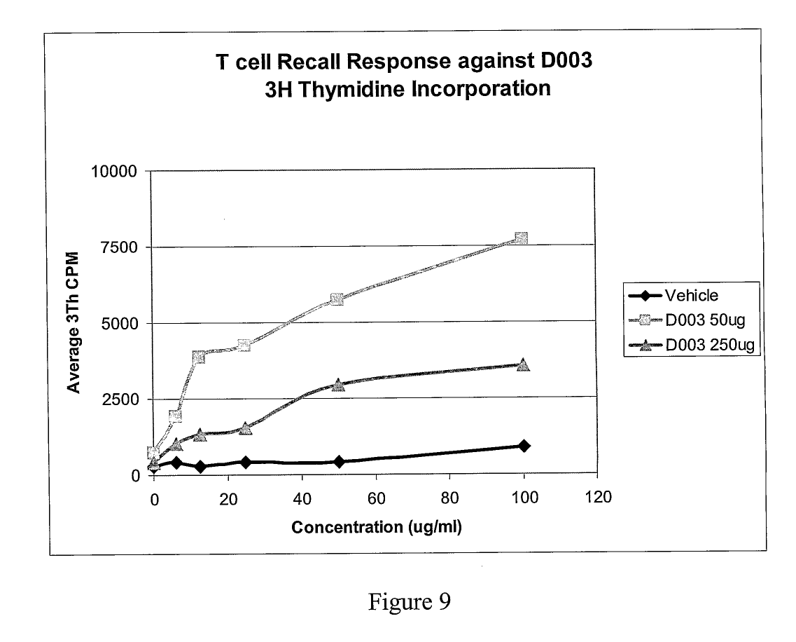

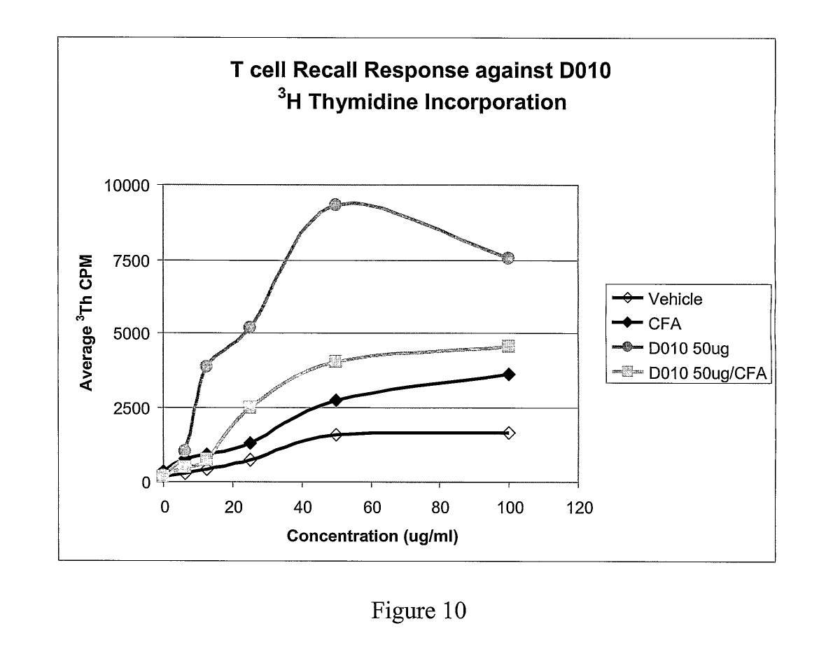

The results of the experiments are shown in FIGS. 7-10. The figures each show the incorporation of 3H Thymidine by the splenocytes from the spleens of mice immunized with RSP-001, 002, 003 and 010 by the copolymers that were used to immunize them. These experiments show semi-random copolymers RSP-001, RSP-002, and RSP-003, and random copolymer RSP-010 (DALE) are immunogenic in that they elicit responses in T cells from mice with prior exposure.

### EXAMPLE 7

### Analysis of GAD65 Peptides

The data to be obtained relate to understanding of presentation of GAD peptides to T cells by DR3 and DQ8 proteins complexed with the peptides, and will provide further information on the DQ8 binding motif from the pool of peptide sequence. Induction of tolerance to autoimmune diseases by administration of copolymers and/or auto antigenic peptides is a topic of great practical as well as theoretical interest. Identification of peptides derived from GAD65 and presented by DR and DQ proteins will allow design of amino acid-based human therapeutics.

Data herein describe isolated naturally processed peptides that are bound to HLA-DQ8. Further work will analyze peptides to be obtained from HLA-DQ8 and HLA-DR3 protein in spleens and lymph nodes of Aβ°/DQ3, DQ8 transgenic mice that develop insulitis and are immunized with GAD65 or display GAD65 autoreactivity spontaneously.

The immune system is able to distinguish foreign molecules from the endogenous or “self” cell components such as proteins. Once a foreign molecule is recognized, the immune system enlists participation of a variety of cells (e.g. B and T cells) and molecules to mount an appropriate response to eliminate them. Autoimmunity occurs when an immune response is mounted against self-components. The protein to be recognized by T cells is cleaved, for example, proteolytically, into small fragments (peptides), which are then associated with major histocompatibility complex (MHC) molecules and transported to the cell surface. Examples of MHC molecules are HLA-DR4, DR3, HLA-DQ2 encoded by DQA1*0501-DQB1*0201 allele or HLA-DQ8 encoded by DQA1*03-DQB1*0302 allele, certain alleles of which have been shown to be associated with the greater risk for insulin-dependent diabetes mellitus (IDDM). IDDM is thought to be a T cell-mediated autoimmune disease in which T cells destroy the insulin-secreting β cells of the pancreatic islets of Langerhans. The peptides derived from glutamic acid decarboxylase (GAD65) an enzyme mainly restricted to brain and β cells found in pancreatic islets, have been implicated in the pathogenesis of the disease.

A humanized mouse model of diabetes in which the mice lack endogenous class II genes and instead transgenically express human HLA-DQ8 and DR3 proteins, is used herein. GAD65 is injected into these transgenic mice which are then immunized with GAD65.

Microscale purification of DR3 and DQ8 proteins will be used to obtain these proteins, using the purification procedure that was optimized above using human B cell lines WT20 and Priess cells. This procedure is used applying the materials from transgenic mice. The peptide pool was fractionated and the sequence of the peptides was analyzed. T cell hybridomas are generated from the spleens and lymph nodes of immunized transgenic mice and tested for their response to these peptides. This material is likely to contain several different peptides. The GAD65 peptides are identified from amino acid sequence data, using sequences to be obtained from several peptides in each pool. Finally, the peptides identified are synthesized and mice are immunized with them to determine whether these are immunogenic.

Presentation of GAD65 peptides to T cells by DR3 and DQ8 proteins provides further information on the DQ8 binding motif, by comparison of amino acid sequences of peptides from the pool. Data obtained is used to design the copolymers herein. The definitive identification of peptides derived from GAD65 and presented by DR and DQ proteins could therefore, have important practical consequences for human therapeutics.

### IV. Equivalents

Contemplated equivalents of the copolymers, subunits and other compositions described above include such materials which otherwise correspond thereto, and which have the same general properties thereof (e.g., biocompatible, antineoplastic), wherein one or more simple variations of substituents are made which do not adversely affect the efficacy of such molecule to achieve its intended purpose. In general, the compounds of the present invention may be prepared by the methods illustrated in the general reaction schemes as, for example, described below, or by modifications thereof, using readily available starting materials, reagents, and conventional synthesis procedures. In these reactions, it is also possible to make use of variants that are in themselves known, but are not mentioned here.

All of the above-cited references are hereby incorporated by reference in their entireties.

1. A copolymer composition that functionally binds to a major histocompatibility complex (MHC) protein HLA-DQ comprising semi-random sequence copolymers having a length of at least about 30 amino acid residues with at least two fixed anchor residues which are separated by 7 amino acid residues, wherein:
(1) the anchor residue is selected from aspartic acid residue (D) and glutamic acid residue (E);
(2) the remainder of the copolymer has a random sequence comprising at least two amino acid residues, one amino acid selected from each amino acid residue group
(a) alanine (A) or glycine (G); and
(b) leucine (L), isoleucine (I), valine (V), methionine (M), threonine (T), serine (S), and cysteine (C);
optionally further comprising proline (P).

2-4. (canceled)

5. A copolymer composition comprising amino acid residues:
(1) aspartic acid, alanine, leucine, and glutamic acid (DALE);
(2) aspartic acid, alanine, isoleucine, and glutamic acid (DAIE);
(3) aspartic acid, alanine, valine, and glutamic acid (DAVE);
(4) aspartic acid, alanine, threonine, and glutamic acid (DATE);
(5) aspartic acid, alanine, serine, glutamic acid (DASE);
(6) aspartic acid, glycine, leucine, and glutamic acid (DGLE);
(7) aspartic acid, glycine, isoleucine, and glutamic acid (DGIE);
(8) aspartic acid, glycine, valine, and glutamic acid (DGVE);
(9) aspartic acid, glycine, threonine, and glutamic acid (DGTE); or
(10) aspartic acid, glycine, serine, glutamic acid (DGSE)
in a random sequence.

6. (canceled)

7. The copolymer composition of claim 5, wherein the molar output ratio of amino acid residues D:A:X:E or D:G:X:E, wherein X is L, I, V, S, or T, is about:
(1) 1:10:3:1;
(2) 1:15:3:1;
(3) 1:25:15:5; or
(4) 1:3:1.5:0.2;
wherein the variability in the molar output ratios comprises a range of about 10% between the different amino acids.

8. (canceled)

9. The copolymer composition of claim 5, wherein the molar input ratio of amino acid residues D:A:X:E or D:G:X:E, wherein X is L, I, V, S, or T, is about:
(1) 1:5:3:1;
(2) 1:25:15:5; or
(3) 1:1:1.5:0.2.

10-17. (canceled)

18. The copolymer composition of claim 1, wherein the HLA-DQ is associated with an autoimmune disease selected from pre-diabetes, diabetes mellitus, celiac disease, unwanted immune response, and allergy.

19-22. (canceled)

23. The copolymer composition of claim 1, wherein the HLA-DQ is HLA-DQ2 (a combination of alleles DQA1*0501-DQB1*0201) or HLA-DQ8 (a combination of alleles DQA1*03-DQB1*0302).

24. A pharmaceutical composition for treatment of an autoimmune disease, comprising a pharmaceutically effective amount of a copolymer composition comprising copolymers that functionally bind to an HLA-DQ molecule associated with the autoimmune disease, and a pharmaceutically acceptable carrier and/or an excipient.

25. The pharmaceutical composition of claim 24, wherein the copolymer composition is the copolymer composition functionally binds to a major histocompatibility complex (MHC) protein HLA-DQ comprising semi-random sequence copolymers having a length of at least about 30 amino acid residues with at least two fixed anchor residues which are separated by 7 amino acid residues, wherein:
(1) the anchor residue is selected from aspartic acid residue (D) and glutamic acid residue (E);
(2) the remainder of the copolymer has a random sequence comprising at least two amino acid residues, one amino acid selected from each amino acid residue group
(a) alanine (A) or glycine (G): and
(b) leucine (L), isoleucine (I), valine (V), methionine (M), threonine (T), serine (S), and cysteine (C);
optionally further comprising proline (P): and
wherein the HLA-DQ is associated with an autoimmune disease selected from pre-diabetes, diabetes mellitus, celiac disease, unwanted immune response, and allergy.

26. The pharmaceutical composition of claim 25, further comprising an additional therapeutically active agent.

27-30. (canceled)

31. The pharmaceutical composition of claim 26, wherein the additional therapeutically active agent is insulin or an immunosuppresant.

32-39. (canceled)

40. A method for treating an autoimmune disease comprising administering to a subject having the autoimmune disease a therapeutically effective amount of a copolymer composition that comprises one or more random sequence copolymers that binds to an HLA-DQ molecule associated with the autoimmune disease.

41. The method of claim 40, wherein said copolymer composition is a copolymer composition functionally binds to a major histocompatibility complex (MHC) protein HLA-DQ comprising semi-random sequence copolymers having a length of at least about 30 amino acid residues with at least two fixed anchor residues which are separated by 7 amino acid residues, wherein:
(1) the anchor residue is selected from aspartic acid residue (D) and glutamic acid residue (E);
(2) the remainder of the copolymer has a random sequence comprising at least two amino acid residues, one amino acid selected from each amino acid residue group
(a) alanine (A) or glycine (G); and
(b) leucine (L), isoleucine (I), valine (V), methionine (M), threonine (T), serine (S), and cysteine (C);
optionally further comprising proline (P); and
wherein the HLA-DQ is associated with an autoimmune disease selected from pre-diabetes, diabetes mellitus, celiac disease, unwanted immune response, and allergy.

42. The method of claim 41, further comprising administering a second therapeutically active agent.

43-46. (canceled)

47. The method according to claim 40, wherein the autoimmune disease is selected from: pre-diabetes, diabetes mellitus celiac disease, unwanted immune response, and allergy.

48-67. (canceled)

68. A method for prophylactically treating a subject at risk of or having pre-conditions for developing an autoimmune disease selected from prediabetes, diabetes mellitus, celiac disease, unwanted immune disease, and allergy, comprising administering the copolymer of claim 18, wherein the onset of the autoimmune disease is delayed or prevented.

69-73. (canceled)

74. The method according to claim 68, wherein the subject or one or more genetically related family members of the subject have high blood glucose or high auto-antibody levels, compared to a control subject that does not have the condition.

75. The method according to claim 68, wherein the subject is a recipient of pancreatic islet transplantation.

76-82. (canceled)

83. A method for identifying a copolymer that is therapeutically effective to treat an HLA-DQ mediated autoimmune disease comprising:
(a) synthesizing a random copolymer of amino acids selected from:
(1) hydrophobic, aliphatic residues (leucine, isoleucine, valine, methionine)
(2) acidic residues (aspartic acid, glutamic acid)
(3) small hydrophilic residues (serine, cysteine, threonine)
(4) small aliphatic residues (alanine, glycine) and
(5) proline.
(b) determining binding of said copolymer to an HLA-DQ molecule;
(c) comparing binding of said copolymer to said HLA-DQ molecule with binding of a known autoantigenic peptide to said HLA-DQ;
(d) selecting said copolymer which binds to said HLA-DQ molecule substantially more strongly than said known autoantigenic peptide; and
(e) determining activation of T-helper cells moderated by said HLA-DQ molecule presenting said copolymer.

84. The method of claim 83, wherein said autoantigenic peptide is selected from:
(1) a peptide comprising amino acid residues 9-23 of human insulin;
(2) a peptide comprising amino acid residues 206-220 of human GAD; and
(3) a peptide comprising amino acid residues 441-460 of human HSP60.

85. The method of claim 84, wherein said HLA-DQ molecule is selected from DQA1 *03-DQB11*0302, DQA1*0501-DQB1*0201, a trans dimer between HLA-DQA1*0501-DQB1*0201 and HLA-DQA1*03-DQB1*0302, DQA1*03/B1*0302, DQB1*0201/DQA1*0501, DQB1*0201 and DQA1*03.

86. The method of claim 83, wherein the copolymer modified for detection, said modification selected from biotinylation and labeling with FITC.

87-96. (canceled)
